(19) United States
(12) Patent Application Publication
(10) Pub. No.: US 2008/0255169 A1
(43) Pub. Date: Oct. 16, 2008

(54) Sulphonamide Derivatives

(76) Inventors: David Smith, Naantali (FI); Anne Marjamaki, Littoinen (FI); Marika Ojala, Raisio (FI); Marjo Pihlavisto, Kaarina (FI); Jyrki Heino, Turku (FI); Jarmo Kapyla, Jyvaskyla (FI); Olli Pentikainen, Lieto (FI); Tommi Nyronen, Helsinki (FI); Mark Johnson, Turku (FI); Mikko Huhtala, Turku (FI)

Correspondence Address: BIRCH STEWART KOLASCH & BIRCH, PO BOX 747, FALLS CHURCH, VA, 22040-0747, US

(21) Appl. No.: 10/593,186

(22) PCT Filed: Jul. 12, 2004
(86) PCT No.: PCT/FI2004/000447

§ 371 Date: Oct. 16, 2006

(30) Foreign Application Priority Data
Mar. 19, 2004 (FI) 2004/000160

(51) Int. Cl. A61K 31/18; C07C 311/21; C07D 209/88; C07D 333/34; C07D 239/42; C07D 277/62; C07D 209/08; C07D 207/335; C07D 263/54; C07D 213/52; A61K 31/505; A61K 31/44; A61K 31/428; A61K 31/421; A61K 31/4164; A61K 31/404

(52) U.S. Cl. 514/275; 564/84; 564/86; 548/444; 549/65; 544/332; 548/178; 548/469; 548/560; 548/217; 546/329; 514/357; 514/367; 514/374; 514/396; 514/415; 514/602

(57) ABSTRACT

The invention relates to sulphonamide derivatives of formula (I),

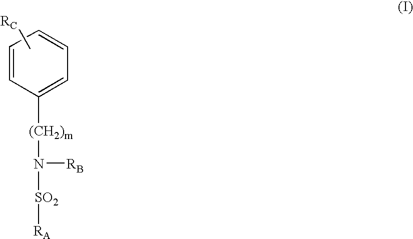

where
R_{C} is optionally substituted 4-6-membered heterocyclic ring containing one or more N atoms, or
R_{C} forms together with the phenyl ring to which it is attached a benzodioxolyl group, or
R_{C} is —NR¹R²,
R_{A} is a group having the formula

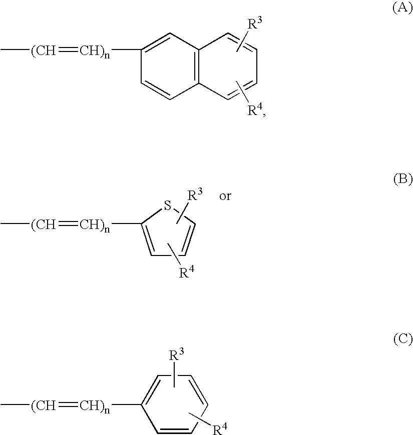

R_{B} is hydrogen or alkyl.

The invention also relates to the use of derivatives of formula (I) as inhibitors for collagen receptor integrins and a process for preparing sulphonamides of formula (I).

### FIELD OF THE INVENTION

The present invention relates to sulphonamide derivatives of formula (I) and physiologically acceptable salts thereof,

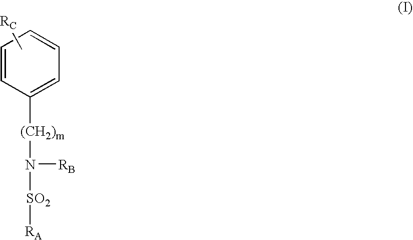

where

R_{C} is an optionally substituted 4-6-membered heterocyclic ring containing one or more N atoms, or

R_{C} forms together with the phenyl ring to which it is attached a benzodioxolyl group, or

R_{C} is —NR¹R², where

R¹ is hydrogen or alkyl,

R² is alkyl or an optionally substituted 4-6-membered heterocyclic ring containing one or more N atoms, or

R¹ and R² taken together with the nitrogen atom to which they are attached form a heterocyclic group, which may contain one or more additional heteroatoms selected from O and N and which may be substituted, or

R¹ and R² are absent and the nitrogen atom together with the adjacent carbon atom forms a heterocyclic ring, which may contain one or more additional heteroatoms selected from N, O and S and which may be substituted,

m is 0 or 1,

R_{A} is a group having the formula

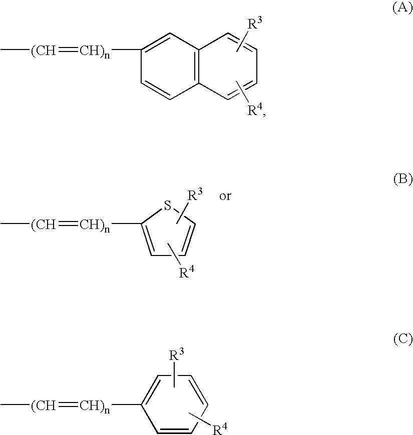

wherein

n is 0 or 1, and

R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl or heterocyclic group, and

R_{B} is hydrogen or alkyl.

The invention also relates to the use of the derivatives of formula (I) as inhibitors of collagen receptor integrins, especially α2β1 integrin inhibitors and more precisely α2β1 integrin I-domain inhibitors, e.g. in connection with diseases and medical conditions that involve the action of cells and platelets expressing collagen receptors, their use as a medicament, e.g. for the treatment of thrombosis and cancer spread, pharmaceutical compositions containing them and a process for preparing them.

### BACKGROUND OF THE INVENTION

The integrins are a large family of cell surface receptors, which mediate cell adhesion to extracellular matrix. They are composed of one α and one β subunit that form a noncovalently bound dimer. In man there are eight β and eighteen α subunits that can form 24 different combinations. Integrins can be divided into three subcategories, namely (i) fibronectin and vitronectin receptors, which recognize an RGD-motif in their ligands, (ii) laminin receptors, and (iii) integrins that have a special inserted-domain (I-domain) in their a sub-unit. The I-domain integrins have been found only in Chordates (includes vertebrates), but not in Nematodes or Arthropods (Hynes et al., J. Cell Biol., 2000, 150:F89-96). Four out of nine I-domain integrins, namely α1β1, α2β1, α10β1 and α11β1 are collagen receptors (Gullberg et al., Prog Histochem Cytochem., 2002, 37:3-54). Collagens are the most abundant extracellular matrix proteins. Twenty-six collagen subtypes (types I-XXVI) are known at the moment (Myllyharju and Kivirikko, 2001, Ann. Med. 33:7-21). In man all four collagen receptor integrins have different expression pattern. Integrin α2β1 is expressed on epithelial cells, platelets, endothelial cells, fibroblasts, chondrocytes (Zutter and Santoro, Am. J. Pathol., 1990, 137:113-120), lymphocytes, mast cells (Kruger-Krasagakes et al., J. Invest. Dermatol., 1996, 106:538-543), and neutrophilic granulocytes (Werr et al., Blood, 2000, 95:1804-1809). Integrin α2β1 deficient knock-out animals are viable, but their platelets do not react to stimulation with collagen (Chen et al., Am. J. Pathol., 2002, 161:337-344; Holtkotter et al., J. Biol. Chem., 2002, 277:10789-10794). In animal models α2β1 also seems to participate in cancer-related angiogenesis (Senger et al., Proc. Natl. Acad. Sci. U.S.A., 1997, 94:13612-13617; Senger et al., Am. J. Pathol., 2002, 160:195-204) and chronic inflammation (de Fougerolles et al., J. Clin. Invest., 2000, 105:721-729). Epidemiological studies have indicated that in man high level of α2β1 integrin on platelet surface is a risk factor for cerebrovascular stroke and myocardial infarction (Moshfegh et al., Lancet, 1999, 353:351-354; Carlsson et al., Blood, 1999, 93:3583-3586). In addition, integrin α2β1 is expressed on variable cancer cell types, and is involved with invasion and progression of melanoma (Klein et al., J. Invest. Dermatol., 1991, 96:281-284), ovarian cancer (Fishman et al., Invasion Metastasis, 1998, 18:15-26), prostate cancer (Bonkhoff et al., Hum. Pathol., 1993, 24:243-248), and gastric cancer (Kawamura et al., Int. J. Oncol., 2001, 18:809-815).

The collagen receptor integrins use their αI-domains in ligand recognition and binding. Human recombinant αI-domains have been used to analyze to molecular details of the binding mechanism (Emsley et al., Cell, 2000, 101:47-56). In all four collagen binding αI-domains (termed as α1I, α2I, α10I, α11I) the basic structure is very similar. However, αI-domain binding assays have indicated that their ligand binding mechanisms and, for example, their ability to bind to different collagen subtypes is different (Gullberg et al., Prog Histochem Cytochem., 2002, 37:3-54).

One known inhibitor of α2I-domain binding is a cyclic compound disclosed in the international patent publication WO 9902551.

It has now surprisingly been found that the compounds of formula (I) according to the present invention are potent inhibitors for collagen receptor integrins, especially α2β1 integrin, and may be used in the treatment of human diseases, such as thrombosis, cancer, fibrosis and inflammation. The compounds of formula (I) may also be used in diagnostic methods both in vitro and in vivo.

### SUMMARY OF THE INVENTION

The present invention relates sulphonamide derivatives of formula (I) and physiologically acceptable salts thereof,

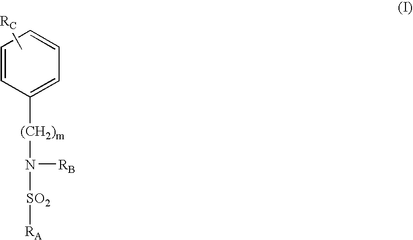

where

R_{C} is an optionally substituted 4-6-membered heterocyclic ring containing one or more N atoms, or

R_{C} forms together with the phenyl ring to which it is attached a benzodioxolyl group, or

R_{C} is —NR¹R², where

R¹ is hydrogen or alkyl,

R² is alkyl, or an optionally substituted 4-6-membered heterocyclic ring containing one or more N atoms, or

R¹ and R² taken together with the nitrogen atom to which they are attached form a heterocyclic group, which may contain one or more additional heteroatoms selected from O and N and which may be substituted, or

R¹ and R² are absent and the nitrogen atom together with the adjacent carbon atom forms a heterocyclic ring, which may contain one or more additional heteroatoms selected from N, O and S and which may be substituted,

m is 0 or 1,

R_{A} is a group having the formula

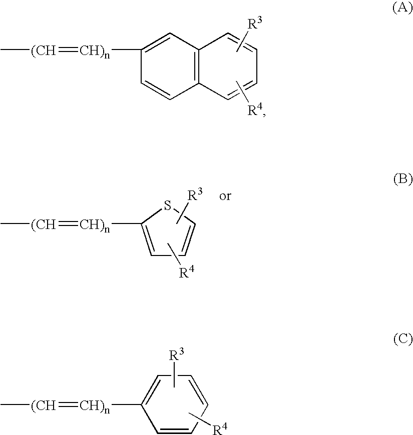

wherein

n is 0 or 1, and

R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl or heterocyclic group, and

R_{B} is hydrogen or alkyl.

Further the invention relates to derivatives of formula (I) for use as inhibitors for collagen receptor integrins specifically α2β1 integrin inhibitors and more precisely α2β1 integrin I-domain inhibitors.

The invention also relates to derivatives of formula (I) and physiologically acceptable salts thereof for use as a medicament.

Further the invention relates to the use of a derivative of formula (I) for preparing a pharmaceutical composition for treating disorders relating to thrombosis and cancer spread.

The present invention also relates to a pharmaceutical composition comprising an effective amount of a derivative of formula (I) or a physiologically acceptable salts thereof in admixture with a pharmaceutically acceptable carrier.

Further the invention relates to a process for preparing benzenesulphonamide derivatives of formula (I) comprising reacting a compound of formula (II),

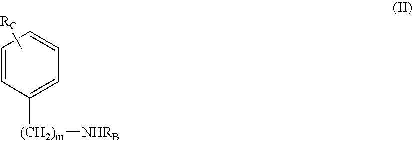

where R_{B}, R_{C} and m are as defined above, with a compound of formula (III),

R_{A}—SO₂hal (III)

where R_{A} is as defined above and hal is halogen.

### DETAILED DESCRIPTION OF THE INVENTION

In the definition of the compound group of formula (I), the meaning of the term “an optionally substituted 4-6-membered heterocyclic ring containing one or more N atoms” for R_{C} is e.g. a group having formula

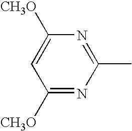

R_{C} may also represent a bivalent group of formula —O—CH₂—O— attached to two adjacent carbon atoms in the phenyl ring thus forming together with the phenyl ring a benzodioxolyl group.

When R_{C} is —NR¹R², the meaning “alkyl” for R¹ and R² refers to branched or straight chain alkyl groups having suitably 1 to 6 carbon atoms, preferably 1 to 3 carbon atoms, specifically methyl.

Examples of the meaning “4-6-membered heterocyclic ring containing one or more N atom” for R² are pyridyl and pyrimidinyl.

Typical examples of heterocyclic groups formed by R¹ and R² together with the N atom to which they are attached are optionally substituted pyrrole and pyrazole groups, e.g.

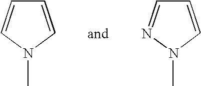

or groups having formulae

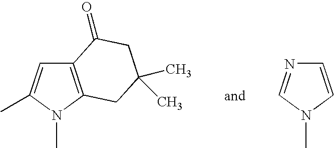

When R¹ and R² are absent the N atom may form together with the adjacent carbon atom in the phenyl ring a fused ring e.g. of formula

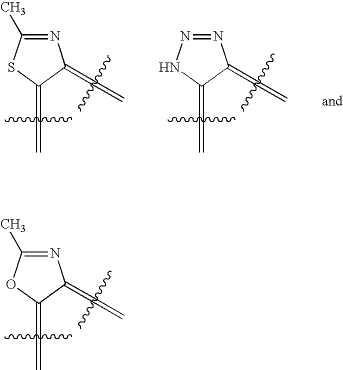

Typical optional substituents in the definition of R_{C} are halogen, alkyl having 1 to 6 carbon atoms, alkoxy having 1 to 6 carbon atoms, halogen and oxo.

In formulae (A), (B) and (C) the meaning of “n” is preferably 0. R³ and R⁴ are suitably halogen, haloaryl or alkoxyaryl. Examples of R³ and R⁴ having the meaning alkoxyalkyl, alkoxycarbonyl and alkanoyl are those containing 1 to 6 carbon atoms in the alkoxy moiety and 1 to 6 carbon atoms in the alkyl moiety. Examples of optionally substituted aryl and heterocyclic groups are

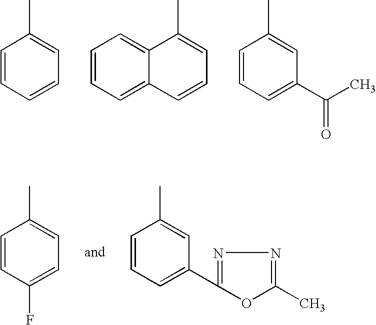

The meaning “alkyl” for R_{B} refers to branched or straight chain alkyl groups having suitably 1 to 6 carbon atoms, preferably 1 to 3 carbon atoms, specifically methyl.

Specific examples of preferred compounds are
3′,4′dimetoxy-biphenyl-3-sulphonic acid (4-dimethylamino-phenyl)-amide),
N-[4-(dimethylamino)phenyl]-4′-fluoro-1′,1′-biphenyl-3-sulphonamide,
2,4-dichloro-N-{4-[(4,6-dimethylpyrimidin-2-yl)(methyl)amino]phenyl}benzenesulphonamide,
N-[4-(dimethylamino)phenyl]-3-(5-methyl-1,3,4-oxadiazol-2-yl)benzenesulphonamide,
2,4-dichloro-N-[4-(2,6,6-trimethyl-4-oxo-4,5,6,7-tetrahydro-1H-indol-1-yl)phenyl]benzenesulphonamide,
2,4-dichloro-N-(2-methyl-1,3-benzothiazoll-5-yl)]benzenesulphonamide,
N-[4-(dimethylamino)phenyl]-4-(1-naphtyl)benzenesulphonamide,
4′-fluoro-biphenyl-3-sulfonic acid benzo[1,3]dioxol-5-ylamide,
4′-fluoro-biphenyl-3-sulfonic acid (2-methyl-benzooxazol-6-yl)-amide,
2,4-dichloro-N-(1,2-dimethyl-1H-indol-5-yl)-N-methyl-benzenesulfonamide,
4′-fluoro-biphenyl-3-sulfonic acid (4-dimethylaminophenyl)-methylamide,
N-[4-(dimethylamino)phenyl]-4′-fluoro-2′-methyl-1,1-biphenyl-3-sulfonamide.

Typical physiologically acceptable salts are e.g. acid addition salts (e.g. HCl, HBr, mesylate, etc.) and alkalimetal and alkaline earth metal salts (Na, K, Ca, Mg, etc.) conventionally used in the pharmaceutical field.

The compounds of formula (I) may be prepared by reacting a compound of formula (II)

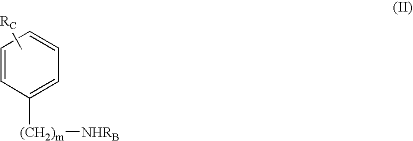

where R_{B}, R_{C} and m are as defined above, with a compound of formula (III)

R_{A}—SO₂hal (III)

where R_{A} is as defined above and hal is halogen.

The reaction may be carried out in conventional manner using methods well-known to the person skilled in the art.

The pharmaceutical compositions can contain one or more of the sulphonamides of the invention. The administration can be parenteral, subcutaneous, intravenous, intraarticular, intrathecal, intramuscular, intraperitoneal or intradermal injections, or by transdermal, buccal, oromucosal, ocular routes or via inhalation. Alternatively or concurrently, administration can be by the oral route. The required dosage will depend upon the severity of the condition of the patient, for example, and such criteria as the patient's weight, sex, age, and medical history. The dose can also vary depending upon whether it is to be administered in a veterinary setting to an animal or to a human patient.

For the purposes of parenteral administration, compositions containing the sulphonamides of the invention are preferably dissolved in distilled water for injection and the pH preferably adjusted to about 6 to 8 and the solution is preferably adjusted to be isotonic. If the sulphonamide is to be provided in a lyophilized form, lactose or mannitol can be added to the solution as a bulking agent and, if necessary, buffers, salts, cryoprotectants and stabilizers can also be added to the composition to facilitate the lyophilization process, the solution is then filtered, introduced into vials and lyophilized.

Useful excipients for the compositions of the invention for parenteral administration also include sterile aqueous and non-aqueous solvents. The compounds of the invention may also be administered parenterally by using suspensions and emulsions as pharmaceutical forms. Examples of useful non-aqueous solvents include propylene glycol, polyethylene glycol, vegetable oil, fish oil, and injectable organic esters. Examples of aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Examples of intravenous infusion vehicles include fluid and nutrient replenishers, electrolyte replenishers, such as those based upon Ringer's dextrose and the like.

Injectable preparations, such as solutions, suspensions or emulsions, may be formulated according to known art, using suitable dispersing or wetting agents and suspending agents, as needed. When the active compounds are in water-soluble form, for example, in the form of water soluble salts, the sterile injectable preparation may employ a non-toxic parenterally acceptable diluent or solvent as, for example, water for injection (USP). Among the other acceptable vehicles and solvents that may be employed are 5% dextrose solution, Ringer's solution and isotonic sodium chloride solution (as described in the Ph. Eur./USP). When the active compounds are in a non-water soluble form, sterile, appropriate lipophilic solvents or vehicles, such as fatty oil, for example, sesame oil, or synthetic fatty acid esters, for example, ethyl oleate or triglycerides, are used. Alternatively, aqueous injection suspensions which contain substances which increase the viscosity, for example, sodium carboxymethyl cellulose, sorbitol, and/or dextran, and optionally also contain stabilizers may be used.

Pharmaceutical preparations for oral (but systemic) administration can be obtained by combining the active compounds with solid excipients, optionally granulating a resulting mixture and processing the mixture or granules or solid mixture without granulating, after adding suitable auxiliaries, if desired or necessary, to give tablets or capsules after filling into hard capsules.

Suitable excipients are, in particular, fillers such as sugars, for example lactose or sucrose, mannitol or sorbitol, cellulose and/or starch preparations and/or calcium phosphates, for example tricalcium phosphate or calcium hydrogen phosphate, as well as binders, such as starches and their derivatives, pastes, using, for example, maize starch, wheat starch, rice starch, or potato starch, gelatine, tragacanth, methyl cellulose, hydroxypropylmethyl cellulose, sodium carboxymethyl cellulose, and/or polyvinyl pyrrolidone, derivatives, and/or, if desired, disintegrating agents, such as the above-mentioned starches, and also carboxymethyl-starch, cross-linked polyvinyl pyrrolidone, agar or alginic acid or a salt thereof, such as sodium alginate. Auxiliaries are, above all, flow-regulating agents and lubricants, for example, silica, talc, stearic acid or salts thereof, such as magnesium stearate or calcium stearate, with suitable coating, which if desired, are resistant to gastric juices and for this purpose, inter alia concentrated sugar solutions, which optionally contain gum arabic, talc, polyvinyl pyrrolidone, polyethylene glycol and/or titanium dioxide, lacquer solutions and suitable organic solvents or solvent mixtures, but also film coating using cellulose derivatives, polyethylene glycols and/or PVP derivatives may be used. In order to produce coatings resistant to gastric juices, solutions of suitable Cellulose preparations such as acetyl cellulose phthalate or hydroxypropylmethyl cellulose phthalate, are used for coating. Dyestuffs or pigments may be added to the tablets or dragee coatings, for example, for identification or in order to characterize different combinations of active compound doses.

Solid dosage forms for oral administration include capsules, tablets, pills, troches, lozenges, powders and granules. In such solid dosage forms, the active compound may be admixed with at least one inert diluent such as sucrose, lactose or starch. Such dosage forms may also comprise, as is normal practice, pharmaceutical adjuvant substances, e.g., stearate lubricating agents or flavouring agents. Solid oral preparations can also be prepared with enteric or other coatings which modulate release of the active ingredients.

Liquid dosage forms for oral administration include pharmaceutically acceptable emulsions, solutions, suspensions, syrups and elixirs containing inert non-toxic diluents commonly used in the art, such as water and alcohol. Such compositions may also comprise adjuvants, such as wetting agents, emulsifying, suspending, sweetening and flavouring agents.

The compositions of the invention may also be administered by means of pumps, or in sustained-release form. The compounds of the invention may also be delivered to specific organs in high concentration by means of suitably inserted catheters, or by providing such molecules as a part of a chimeric molecule (or complex) which is designed to target specific organs.

Administration in a sustained-release form is more convenient for the patient when repeated injections for prolonged periods of time are indicated so as to maximize the comfort of the patient. Controlled release preparation can be achieved by the use of polymers to complex or adsorb the peptides of the invention. Controlled delivery can be achieved by selecting appropriate macromolecules (for example, polyesters, polyamino acids, polyvinyl pyrrolidone, ethylenevinylacetate, methylcellulose, carboxymethylcelluloase protamine zinc and protamine sulfate) as well as the method of incorporation in order to control release. Another possible method to control the duration of action by controlled release preparations is to incorporate the desired peptide into particles of a polymeric material such as polyesters, polyamino acids, hydrogels, poly (lactic acid) or ethylene vinylacetate copolymers. Alternatively, instead of incorporating the sulphonamide into these polymeric particles, the sulphonamide can be entrapped into microparticles, prepared, for example, by coacervation techniques or by interfacial polymerization, for example, hydroxymethylcellulose or gelatin-microcapsules and poly (methylmethacrylate) microcapsules, respectively, or in colloidal drug delivery systems, for example liposomes, albumin microspheres, microemulsions, nanoparticles, and nano-capsules or in macroemulsions. The above-mentioned technics may be applied to both parenteral and oral administration of the pharmaceutical formulation.

The sulphonamides that are used in the compositions and methods of the invention can be employed in dosage forms such as tablets, coated tablets, capsules, powder sachets, or liquid solutions for oral administration if the biological activity of the material is not destroyed by the digestive process and if the characteristics of the compound allow it to be absorbed across the intestinal tissue.

The pharmaceutical compositions of the present invention can be manufactured in a manner which is in itself know, for example, by means of conventional mixing, granulating, dragee-making, dissolving, lyophilizing or similar processes.

The compounds of the invention are potent collagen receptor inhibitors and useful for inhibiting or preventing the adhesion of cells on collagen or the migration and invasion of cells through collagen, in vivo or in vitro. The now described compounds inhibit the migration of malignant cells and are thus for treating diseases such as cancers, including prostate, and melanoma, especially where α2β1 integrin dependent cell adhesion/invasion/migration may contribute to the malignant mechanism.

The compounds of the invention also inhibit adhesion of platelets to collagen and collagen-induced platelet aggregation. Thus, the compounds of the invention are useful for treating patients in need of preventative or ameliorative treatment for conditions or diseases such as cardiovascular diseases that are characterized by a need to prevent adhesion of platelets to collagen and collagen-induced platelet aggregation, for example, in stroke victims or patients at risk of having a stroke.

### Pharmacological Tests

### a Cell Invasion Assay was Used to Demonstrate the Anti-Cancer Potential of the Inhibitors In Vitro

The ability to interact with extracellular matrix basement membranes is essential for the malignant cancer cell phenotype and cancer spread. α2β1 levels are known to be upregulated in tumorigenic cells. The overexpression regulates cell adhesion and migration to and invasion through the extracellular matrix. By blocking the interaction between extracellular matrix components like collagen and α2β1 it is possible to block cancer cell migration and invasion in vitro. Prostate cancer cells (PC-3) expressing α2β1 endogenously were used to test the in vitro anticancer potential of the inhibitors of the present invention.

### Experimental Procedure

Invasion of PC-3 cells (CRL-1435, ATCC) through Matrigel was studied using BD Biocoat invasion inserts (BD Biosciences). Inserts were stored at −20° C. Before the experiments inserts were allowed to adjust to the room temperature. 500 μl of serum free media (Ham's F12K medium, 2 mM L-glutamine, 1.5 g/l sodium bicarbonate) was added into the inserts and allowed to rehydrate at 37° C. in cell incubator for two hours. The remaining media was aspirated. PC-3 cells were detached, pelleted and suspended into serum free media (50 000 cells/500 μl). 300 μl of cell suspension was added into the insert in the absence (control) or presence of the inhibitor according to the pre-sent invention. Inserts were placed on the 24-well plates; each well containing 700 μl of cell culture media with 3% of fetal bovine serum as chemo-attractant. Cells were allowed to invade for 72 hours at 37° C. in cell incubator. Inserts were washed with 700 μl PBS, and fixed with 4% paraformaldehyde for 10 minutes. Paraformaldehyde was aspirated and cells were washed with 700 μl of PBS and inserts were stained by incubation with hematoxylin for 1 minute. The stain was removed by washing the inserts with 700 μl of PBS. Inserts were allowed to dry. Fixed invaded cells were calculated under the microscope. Invasion % was calculated as a comparison to the control.

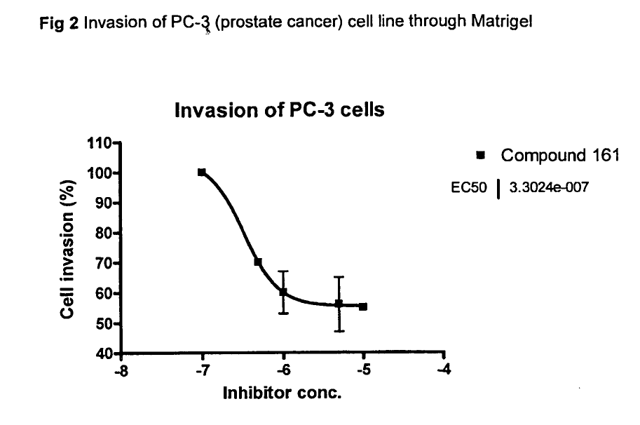

Cell invasion assay is used as an in vitro cancer metastatis model. The sulfonamide molecules have been shown to inhibit tumor cell invasion in vitro. Some structures inhibit invasion even with submicromolar concentrations. Such molecules include compounds 131, 161, 176, 183, 222, 239, 242, 281, 285, 298 (see Table 1 below) and (EC50 is ≦1 μm). In FIG. 2, the dose response of compound 161 in invasion assay is shown. Compound 161 gave the best EC50 value (0.3 μM) in invasion assay. Invasion assay was done with human prostata cancer cell line, PC-3.

### A Platelet Function Analyzer PFA100 was Used to Demonstrate the Anti-Thrombotic Potential of the α2β1 Inhibitors

A platelet function analyzer PFA 100 was used to demonstrate the possible antithrombotic effects of α2β1 inhibitors. The PFA 100 is a high shear-inducing device that simulates primary hemostasis after injury of a small vessel. The system comprises a test-cartridge containing a biologically active membrane coated with collagen plus ADP. An anticoaculated whole blood sample was run through a capillary under a constant vacuum. The platelet agonist (ADP) on the membrane and the high shear rate resulted in activation of platelet aggregation, leading to occlusion of the aperture with a stable platelet plug. The time required to obtain full occlusion of the aperture was designated as the “closure time”. Each hit compound was added to the whole blood sample and the closure time was measured with PFA 100. If the closure time was increased when compared to the control sample the hit compound was suggested to have antithrombotic activity.

### Experimental Procedure

Blood was collected from a single donor via venipuncture into evacuated blood collection tubes containing lithium heparin as anticoagulant. Within 30 minutes, blood was aliquoted into 50 mL falcon tubes and treated with either inhibitory compounds (e.g. mAbs P1H5, 5E8, P1E6) or, as controls, non-specific rat IgG or PBS only at pH 7.4. All experimental and control compounds were diluted in PBS before addition to 0.5% total volume (i.e. 15.92 mL blood and 80 μl compound in PBS). Samples were kept at room temperature with rotation for the duration of the experiments. Duplicate sample volumes (800 μl) were dispensed into PFA Collagen/ADP cartridges, and individual closure times were determined.

Control and experimental samples were tested in two or three sequences during the interval of 60 to 180 minutes from draw. This practice allowed the observation of increasing inhibitory effects over time.

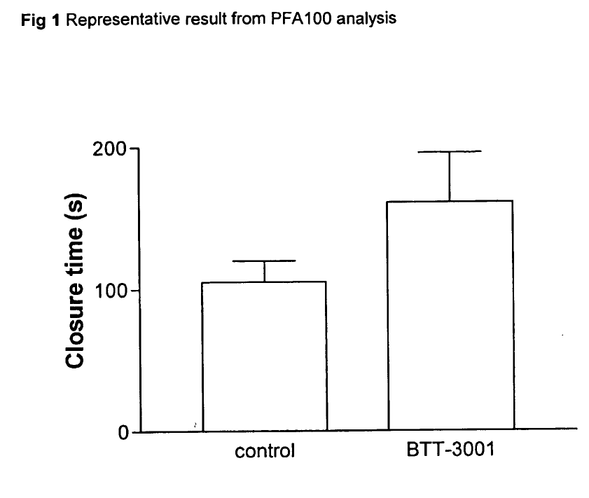

Acquisitions resulting in a closure time exceeding the range of measurement of the instrument (>300 seconds) were assigned a value of 300 seconds. Mean and standard deviations were calculated for each treatment, and data points falling outside ±2 SD of the mean were excluded. Student's t-test was applied to the resultant data. The results are presented in attached FIG. 1.

FIG. 1 contains results with coded compound BTT-3001 (compound 50)=2,4-dichloro-N-{4-[(4,6-dimethylpyrimidin-2-yl)(methyl)amino]-phenyl}benzenesulphonamide.

Further, the compounds listed in Table 1 below were tested. The representative results for active compounds are presented in Table 2.

TABLE 1

| | Compound |
|---|---|
| | number |
| 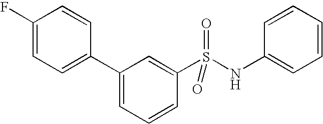 | 324 |
| 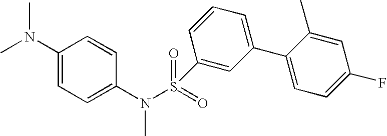 | 321 |
| 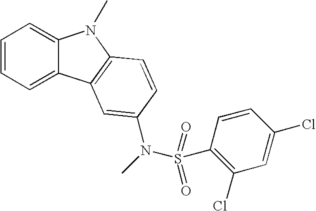 | 320 |
| 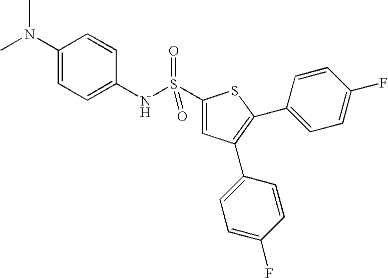 | 317 |
| 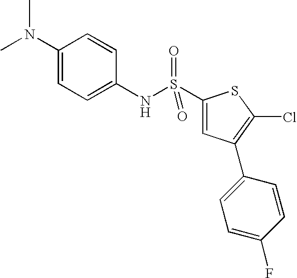 | 316 |
| 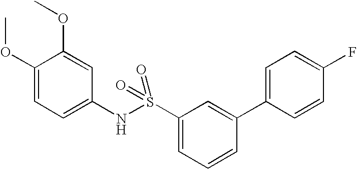 | 310 |
| 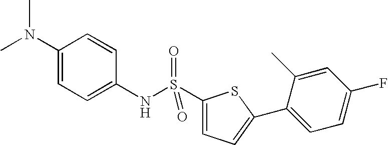 | 307 |
| 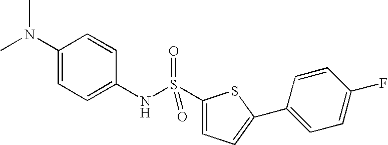 | 306 |
| 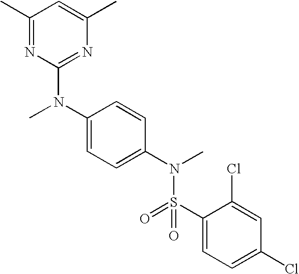 | 302 |
| 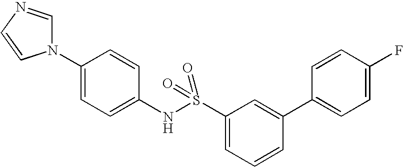 | 300 |
| 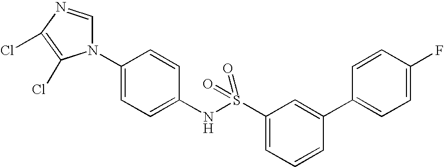 | 299 |
| 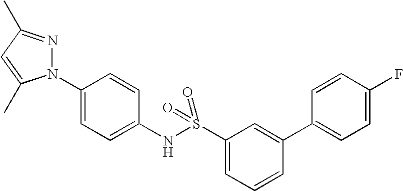 | 298 |
| 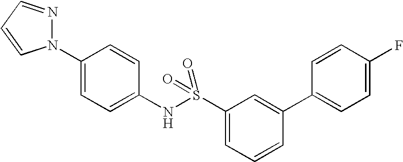 | 297 |
| 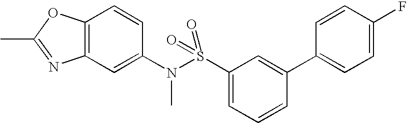 | 295 |
| 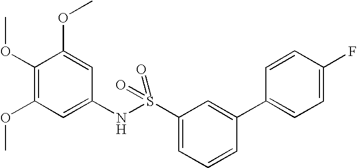 | 294 |
| 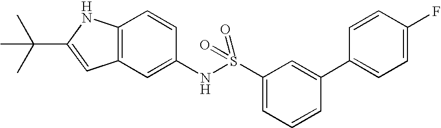 | 291 |
| 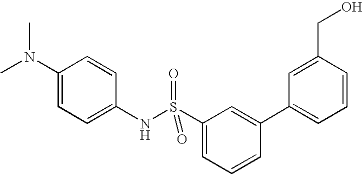 | 288 |
| 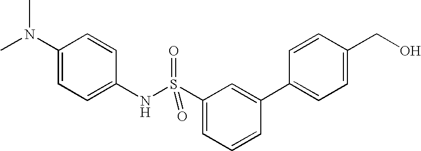 | 287 |
| 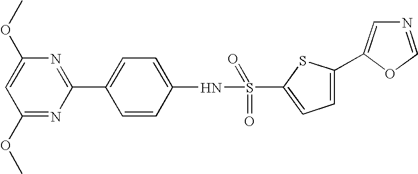 | 286 |
| 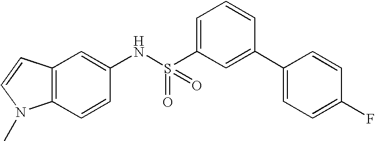 | 285 |
| 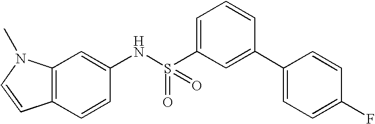 | 284 |
| 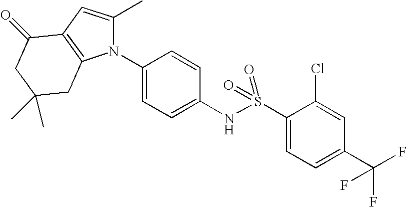 | 283 |
| 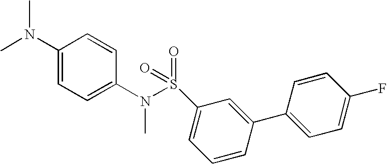 | 282 |
| 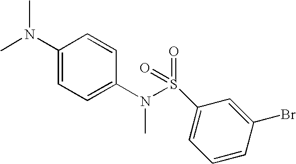 | 281 |
| 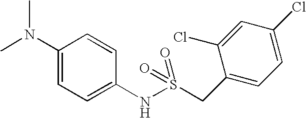 | 278 |
| 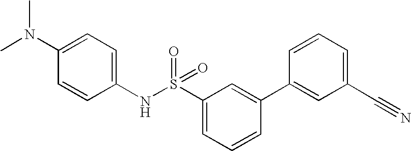 | 275 |
| 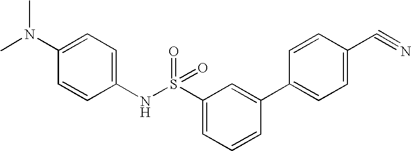 | 274 |
| 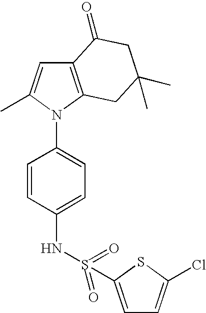 | 271 |
| 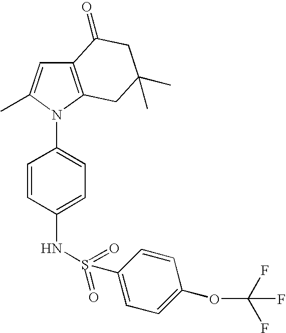 | 270 |
| 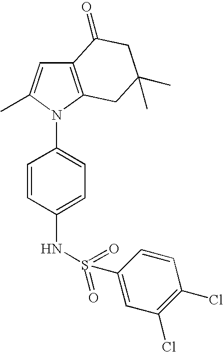 | 269 |
| 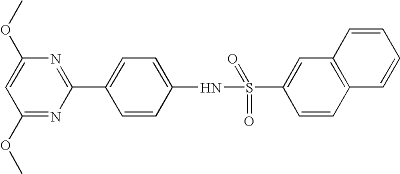 | 266 |
| 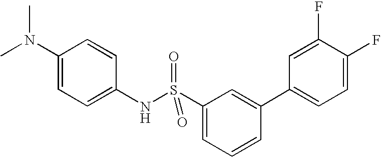 | 263 |
| 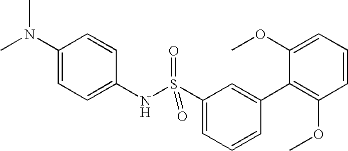 | 262 |
| 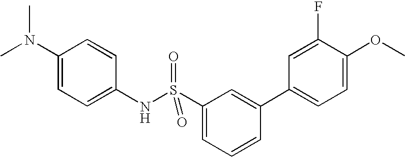 | 261 |
| 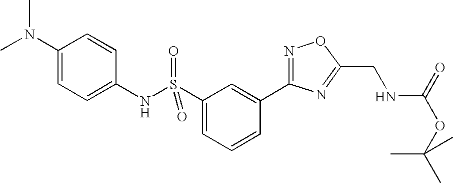 | 260 |
| 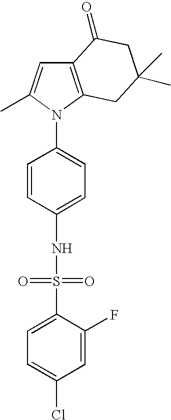 | 259 |
| 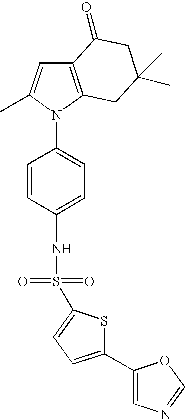 | 258 |
| 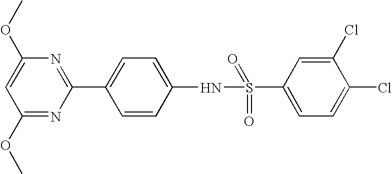 | 255 |
| 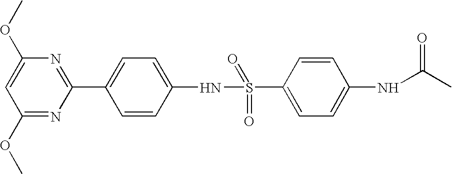 | 254 |
| 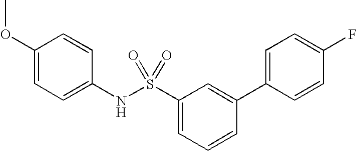 | 250 |
| 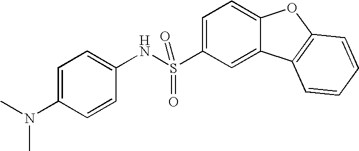 | 249 |
| 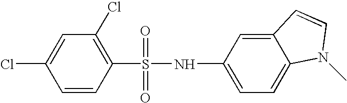 | 248 |
| 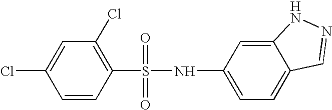 | 247 |
| 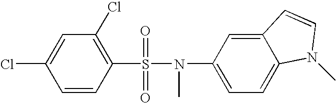 | 244 |
| 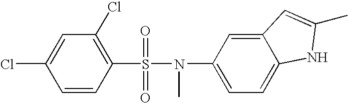 | 243 |
| 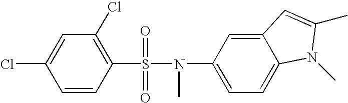 | 242 |
| 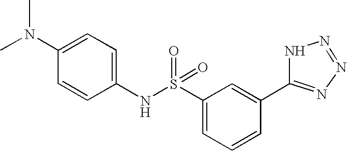 | 241 |
| 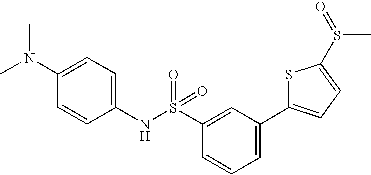 | 239 |
| 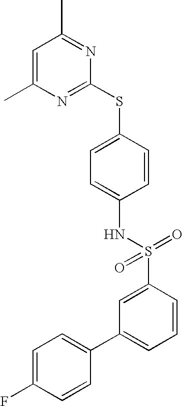 | 238 |
| 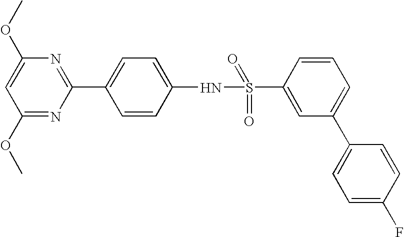 | 235 |
| 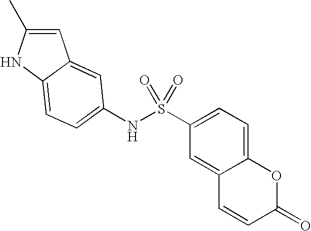 | 234 |
| 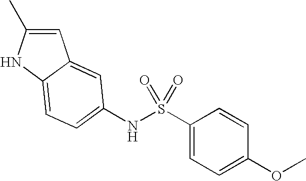 | 233 |
| 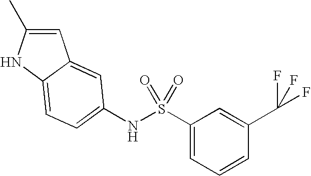 | 230 |
| 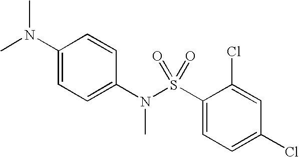 | 223 |
| 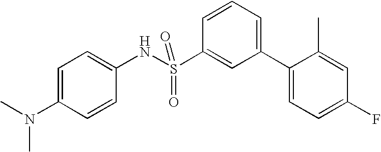 | 222 |
| 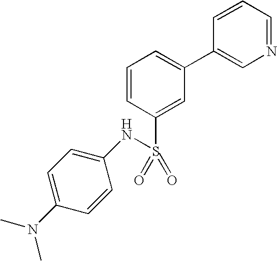 | 220 |
| 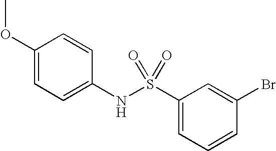 | 216 |
| 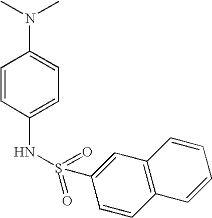 | 214 |
| 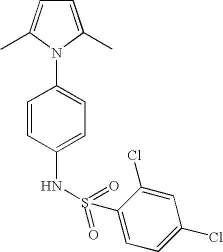 | 213 |
| 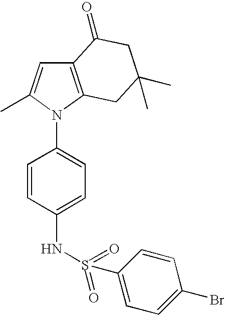 | 212 |
| 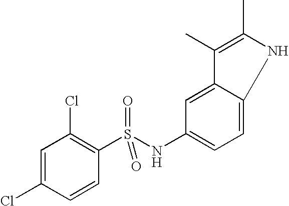 | 210 |
| 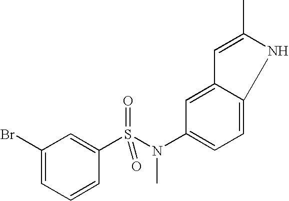 | 209 |
| 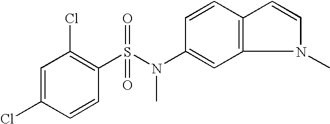 | 208 |
| 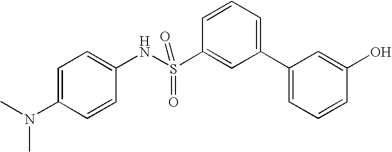 | 205 |
| 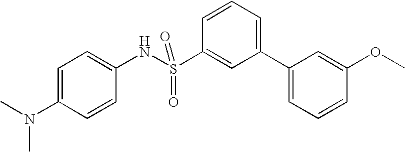 | 204 |
| 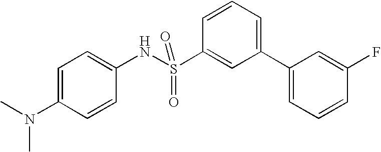 | 203 |
| 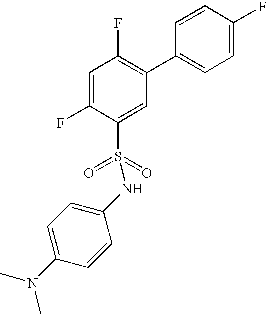 | 202 |
| 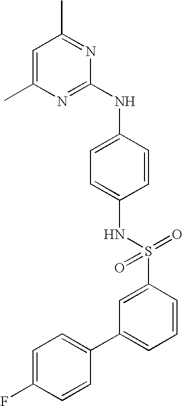 | 201 |
| 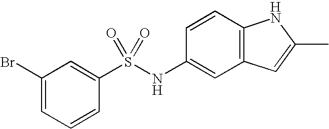 | 197 |
| 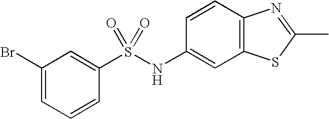 | 195 |
| 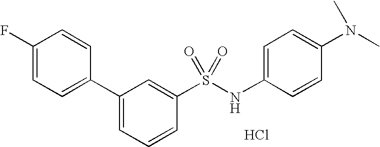 | 193 |
| 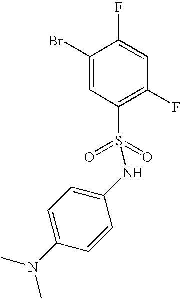 | 192 |
| 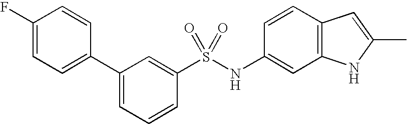 | 191 |
| 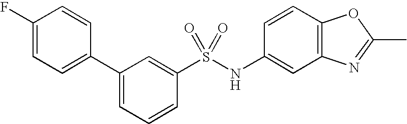 | 190 |
| 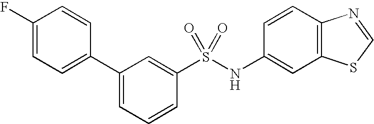 | 189 |
| 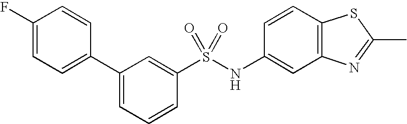 | 188 |
| 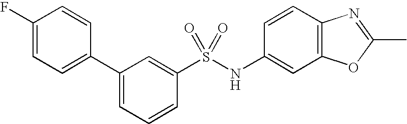 | 187 |
| 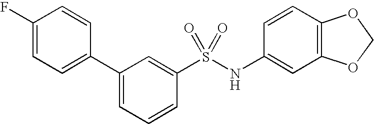 | 186 |
| 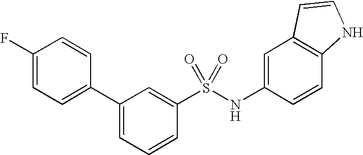 | 184 |
| 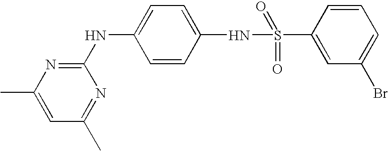 | 183 |
| 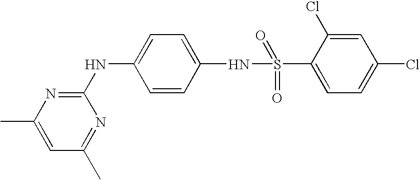 | 182 |
| 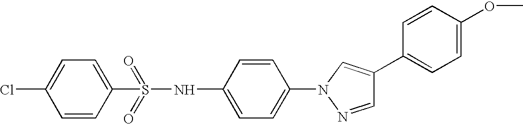 | 176 |
| 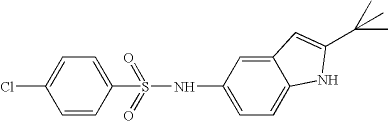 | 173 |
| 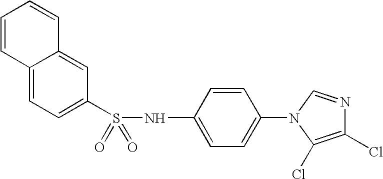 | 171 |
| 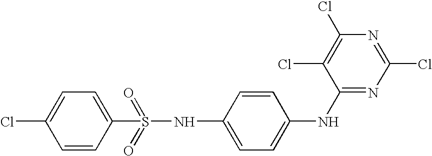 | 170 |
| 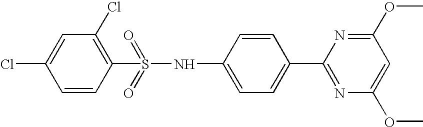 | 164 |
| 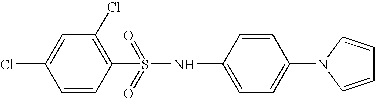 | 163 |
| 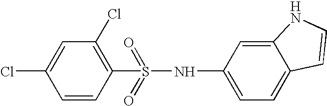 | 161 |
| 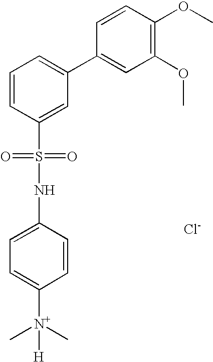 | 142 |
| 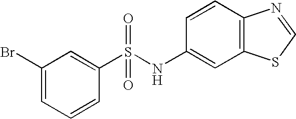 | 139 |
| 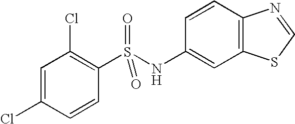 | 138 |
| 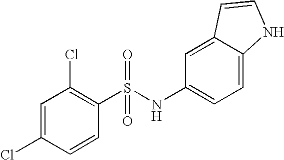 | 134 |
| 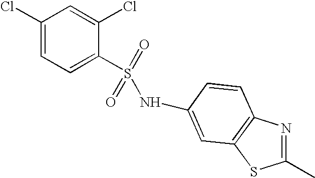 | 133 |
| 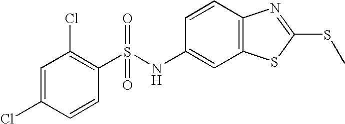 | 132 |
| 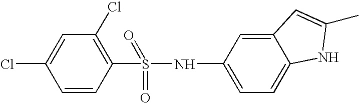 | 131 |
| 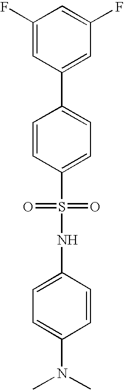 | 124 |
| 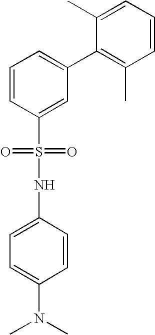 | 122 |
| 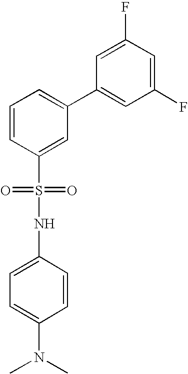 | 120 |
| 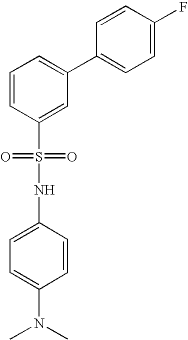 | 119 |
| 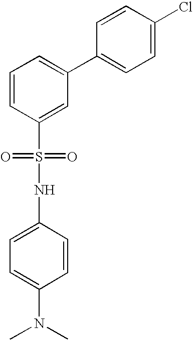 | 118 |
| 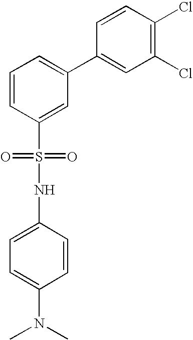 | 114 |
| 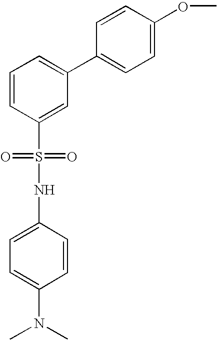 | 112 |
| 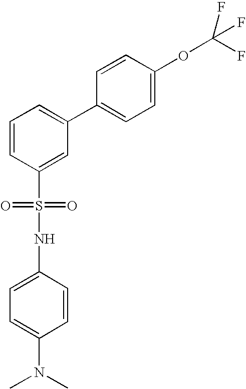 | 107 |
| 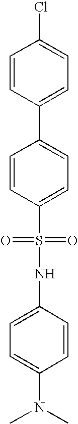 | 103 |
| 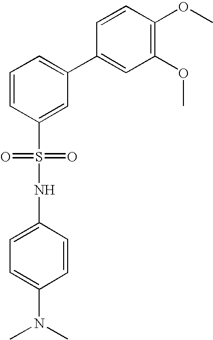 | 102 |
| 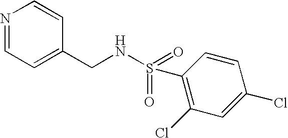 | 98 |
| 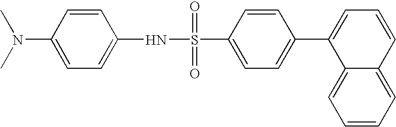 | 90 |
| 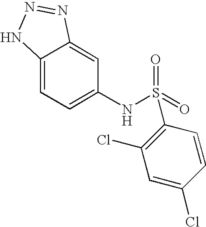 | 87 |
| 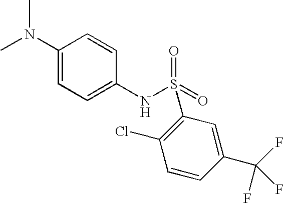 | 85 |
| 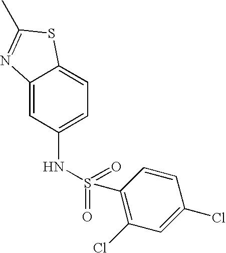 | 84 |
| 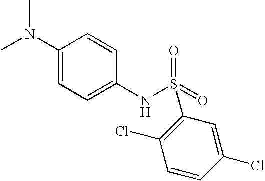 | 83 |
| 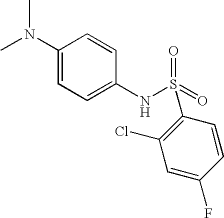 | 81 |
| 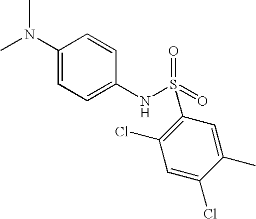 | 80 |
| 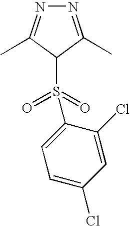 | 78 |
| 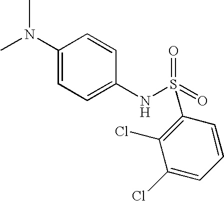 | 77 |
| 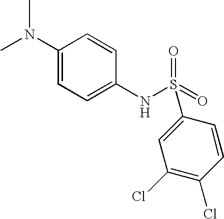 | 76 |
| 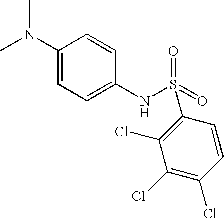 | 75 |
| 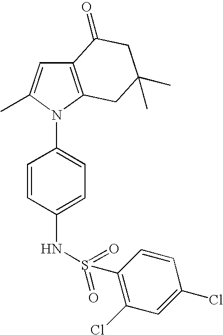 | 71 |
| 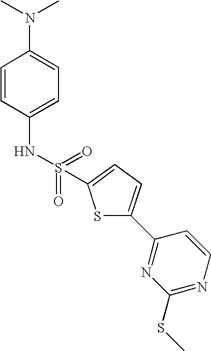 | 65 |
| 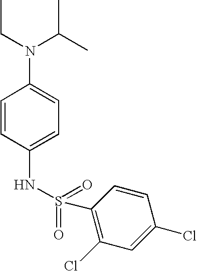 | 64 |
| 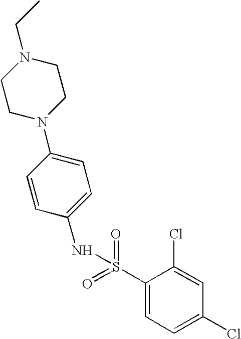 | 63 |
| 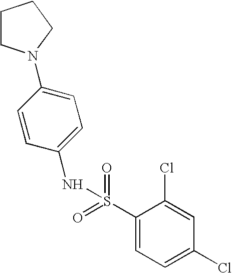 | 62 |
| 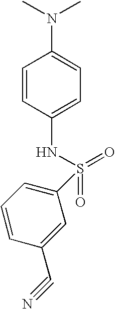 | 60 |
| 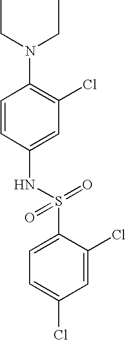 | 52 |
| 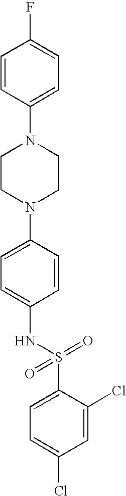 | 51 |
| 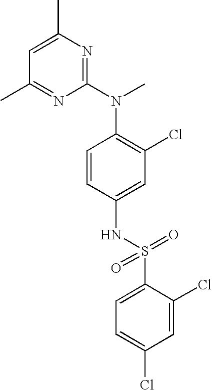 | 50 |
| 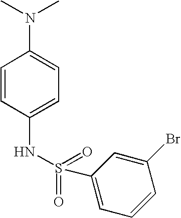 | 49 |
| 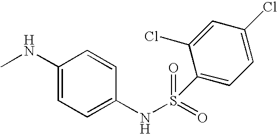 | 38 |
| 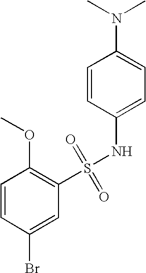 | 33 |

TABLE 2

| | | |
|---|---|---|
| Compound | EC50 in cell adhesion | Emax in cell adhesion |
| number | assay | assay |

| 50 | 17 | μM | 60% |
|---|---|---|---|
| 102 | 13 | μM | 65% |
| 119 | 3 | μM | 70% |
| 131 | 25 | μM | 58% |
| 132 | 40 | μM | 90% |
| 134 | 10 | μM | 40% |
| 139 | 46 | μM | 75% |
| 142 | 20 | μM | 77% |
| 161 | 34 | μM | 82% |
| 163 | 32 | μM | 66% at 50 μM |
| 164 | 21 | μM | 85% at 50 μM |
| 170 | 24 | μM | 85% at 40 μM |
| 171 | 20 | μM | 79% |
| 173 | 35 | μM | 59% at 40 μM |
| 176 | 17 | μM | 59% at 50 μM |
| 182 | 25 | μM | 77% |
| 183 | 28 | μM | 81% |
| 186 | 19 | μM | 91% |
| 187 | 18 | μM | 87% |
| 188 | 36 | μM | 81% |
| 189 | 30 | μM | 76% |
| 190 | 25 | μM | 76% |
| 192 | 39 | μM | 75% |
| 193 | 22 | μM | 72% |
| 195 | 49 | μM | 60% |
| 197 | 30 | μM | 74% |
| 202 | 27 | μM | 91% |
| 203 | 19 | μM | 86% |
| 204 | ~25 μM (could not be | | 63% |
| | defined by Prism) | | |
| 205 | 20 | μM | 84% (50 μM) |
| 209 | 35 | μM | 64% (50 μM) |
| 210 | dd could not be detected | | 80% (50 μM) |
| 213 | 25 | μM | 71% (50 μM) |
| 201 | 36 | μM | 64% (50 μM) |
| 222 | 15 | μM | 66% |
| 223 | 13 | μM | 82% |
| 230 | >30 μM (could not be | | 76% (at 50 μM) |
| | defined by Prism) | | |
| 234 | 35 | μM | 85% |
| 235 | 20 | μM | 85% |
| 239 | 24 | μM | 64% (at 50 μM) |
| 242 | 6 | μM | 70% |
| 250 | 31 | μM | 89% |
| 255 | 17 | μM | 88% (at 50 μM) |
| 258 | 40 | μM | 66% |
| 263 | 26 | μM | 88% |
| 266 | 18 | μM | 70% |
| 269 | 19 | μM | 64% |
| 275 | 26 | μM | 57% |
| 281 | 47 | μM | 78% |
| 282 | 7 | μM | 59% (at 50 μM) |
| 283 | 23 | μM | 63% |
| 284 | ~30 | μM | 69% |
| 285 | 20 | μM | 60% (at 50 μM) |
| 286 | 37 | μM | 72% |
| 291 | 32 | μM | 50% |
| 295 | 29 | μM | 56% |
| 297 | 26 | μM | 80% |
| 298 | 33 | μM | 79% |
| 299 | 9.6 | μM | 79% |
| 302 | 24 | μM | 57% |
| 306 | 24 | μM | 67% (at 50 μM) |
| 307 | 20 | μM | 67% (at 50 μM) |
| 300 | 45 | μM | 50% |
| 316 | 10 | μM | 87% (at 50 μM) |
| 317 | 44 | μM | 45% |
| 320 | 10 | μM | 45% |
| 321 | 6.3 | μM | 55% |

The test results showed that the compounds of the present invention have an anti-cancer and antithrombotic activity in vitro.

The following examples illustrate the invention but are not intended to limitate the scope of the invention.

### EXAMPLE 1

### 3-bromo-N-[4-(dimethylamino)phenyl]benzenesulphonamide

To a solution of 4-dimethylamino aniline (2 g, 0.0147 mol) and triethylamine (2.25 mL, 0.0162 mol, 1.1 eq.) in acetonitrile (20 mL) at 0° C. under nitrogen was added dropwise a solution of 3-bromobenzene sulphonyl chloride (3.94 g, 0.0154 mol, 1.05 eq.) in acetonitrile (5 mL). The mixture was allowed to warm to room temperature and stirred for 18 hours. The solvent was removed in vacuo and the residue redissolved in ethyl acetate (100 mL). The organic layer was washed with sat aqueous NaHC—O₃ (2×200 mL), water (2×200 mL), brine (200 mL), dried (Na₂SO₄), filtered and concentrated. The product was obtained as a brown solid (3.5 g, 67.0%) and was not purified further.

¹H NMR (300 MHz d₆ DMSO) δ 7.78-7.76 (s, 2ff), 7.61-7.58 (d, 1H), 7.48-7.43 (t, 1H), 6.84-6.80 (d, 2H), 6.57-6.54 (d, 2H), 2.78 (s, 6H); ¹³C NMR (300 MHz d₆ DMSO) δ 148.84, 142.15, 135.70, 131.65, 129.46, 126.12, 125.66, 124.77, 122.24, 112.95; LCMS Rₜ 15.44 min.; m/z—353.3. MP 187-189° C.

### EXAMPLE 2

### 3′,4′-dimethoxy-biphenyl-3-sulphonic acid (4-dimethylamino-phenyl)-amide (BTT-3002=compound 102)

To a solution of 3-bromo-N-(4-dimethylamino-phenyl)-benzenesulphonamide (2.14 g, 6.02 mmol) and 3,4-dimethoxyphenyl-boronic acid (1.09 g, 6.02 mmol) in toluene (200 mL) and aqueous sodium carbonate solution (2 M, 100 mL) under N₂ was added tetrakis(triphenylphosphine) palladium (0) (80 mg). The mixture was stirred under reflux for 18 hours. The reaction mixture was then filtered through celite and washed with ethyl acetate. The organic layer was separated and dried (MgSO₄). After evaporation of the solvent the crude material was purified by column chromatography (SiO₂, ethylacetate/cyclohexane=4/6) to yield 1.8 g (73%) of compound 102 as light yellow crystals: mp 43° C.

¹H NMR (300 MHz, CDCl₃) 8.2.93 (6H, s), 3.94 (6H, s), 6.19 (1H, bs), 6.60 (2H, d, J=9 Hz), 6.9 (4H, m), 7.09 (1H, d, J=9 Hz), 7.46 (1H, t, J=8.8 Hz), 7.64 (1H, d, J=9 Hz), 7.5 (1H, d, J=8.8 Hz, CH), 7.87 (1H, s); ¹³C NMR (300 MHz, CDCl₃) δ40.88, 56.42, 56.45, 110.77, 112.01, 113.04, 120.05, 124.97, 125.76, 125.87, 126.80, 129.59, 131.18, 132.61, 140.21, 142.15, 149.76, 149.81; MS (ES⁺) m/z 413.5 (M+H).

### EXAMPLE 3

### N-[4-(dimethylamino)phenyl]-4′-fluoro-1,1′-biphenyl-3-sulphonamide (BTT-3003=compound 119)

Crude compound of example 1 (3.98 g, 11.2 mmol), 4-fluorobenzene boronic acid (1.57 g, 11.2 mmol) and tetrakis(triphenylphosphine)palladium (160 mg, 0.14 mmol) were stirred in toluene (150 mL, degassed) and 2M sodium bicarbonate solution (100 mL, degassed) at 106° C. overnight. After this time the reaction mixture was filtered through celite, the organic solution separated from the aqueous, which was washed with ethyl acetate and the organic solvents combined. The crude dark brown/black material was decolourised with activated charcoal and recrystallised from isopropanol to give the product (1.8324 g, 44%) as an off white/beige material: mp 158-160° C.

¹H NMR (CDCl₃) δ 3.03 (s, 6H), 6.69 (s, 1H), 6.72 (s, 2H), 7.05-7.08 (d, J=9 Hz, 2H), 7.19-7.24 (t, J=8.7 Hz, 2H), 7.52-7.62 (m, 3H), 7.79-7.81 (m, 2H), 7.94-7.95 (m, 1H); ¹³C NMR (CDCl₃) δ 40.93, 113.14, 116.085, 116.372, 125.02, 126.23, 126.71, 129.28, 129.73, 131.35, 135.81, 140.25, 141.30, 149.86, 161.64, 164.93, LCMS Rf=15.0 mins, (ES)=m/z 371.3 (M+1).

### EXAMPLE 4

### 2,4-dichloro-N-{4-[(4,6-dimethylpyrimidin-2-yl)(methyl)amino]phenyl}benzenesulphonamide (BTT-3001=compound 50)

To a solution of N-(4,6-dimethylpyrimidin-2-yl)-N-methylbenzene-1,4-diamine (2 g, 0.0088 mol) and triethylamine (1.35 mL, 0.0097 mol, 1.1 eq.) in acetonitrile (30 mL) at 0° C. under nitrogen was added dropwise a solution of 2,4-dichlorobenzene sulphonyl chloride (2.26 g, 0.0092 mol, 1.05 eq.) in acetonitrile (10 mL). The mixture was allowed to warm to room temperature and stirred for 18 hours. The solvent was removed in vacuo and the residue redissolved in ethyl acetate (100 mL). The organic layer was washed with sat aqueous NaHCO₃ (2×100 mL), water (2×100 mL), brine (100 mL), dried (Na₂SO₄), filtered and concentrated. The residue was purified by column chromatography (1:4 AcOEt:cyclohexane) to yield 1.26 g of a yellow oil (bis sulphonamide) and 1.77 g (46.2%) of a light green solid (monosulphonamide).

Bis sulphonamide: ¹H NMR (300 MHz CDCl₃) δ 8.13-8.10 (d, 2H), 7.52-7.51 (d, 2H), 7.43-7.38 (dd, 1H), 7.37-7.34 (d, 2H), 7.26-7.22 (d, 2H), 6.43 (s, 1H), 3.56 (s, 3H), 2.29 (s, 6H).

Monosulphonamide: ¹H NMR (300 MHz CDCl₃) δ 7.88-7.85 (d, 1H), 7.46 (d, 1H), 7.26-7.22 (dd, 1H), 7.18-7.14 (d, 2H), 7.01-6.98 (d, 2H), 6.87 (s, NH), 6.29 (s, 1H), 3.40 (s, 3H), 2.18 (s, 6H); ¹³C NMR (300 MHz CDCl₃) δ 167.35, 144.51, 140.29, 135.53, 133.39, 131.78, 131.59, 127.97, 126.99, 123.02, 110.88, 38.46, 24.39; LCMS Rₜ 18.71 min.; m/z—437.4.

### EXAMPLE 5

### Hydrolysis of the 2,4-dichloro-N-[(2,4-dichlorophenyl)sulphonyl]-N-{4-[(4,6-dimethylpyrimidin-2-yl)(methyl)amino]phenyl}benzenesulphonamide

To a solution of the bis sulphonamide (1.26 g, 0.002 mol) in ethanol (50 mL) was added NaOEt (653 mg, 0.0097 mol, 5 eq.) and the reaction was heated to 65° C. for 5 hrs. The solvent was removed in vacuo and residue dissolved in water. The aqueous layer was washed twice with CHCl₃ (50 mL). The organic layers were combined, dried (Na₂SO₄), filtered and concentrated. The solid was purified by column chromatography (1:4-2:3 AcOEt:cyclohexane) to yield a beige solid (550 mg, 64.7%, 2,4-dichloro-N-{4-[(4,6-dimethylpyrimidin-2-yl)(methyl)amino]phenyl}benzenesulphonamide).

¹H NMR (100 MHz CDCl₃) δ 7.88-7.87 (d, 1H), 7.46 (d, 1H), 7.26-7.22 (dd, 1H), 7.18-7.14 (d, 2H), 7.02-6.97 (d, 2H), 6.90 (s, NH), 6.28 (s, 1H), 3.40 (s, 3H), 2.17 (s, 6H); ¹³C NMR (300 MHz CDCl₃) δ 167.36, 144.49, 140.28, 135.54, 133.39, 131.72, 131.61, 127.97, 127.00, 123.00, 110.88, 38.47, 24.39; LCMS R_{T} 18.71 min.; m/z—437.4.

LCMS conditions: 0-97% acetonitrile in water, C18, electrospray+ve.

### EXAMPLE 6

### N-[4-(dimethylamino)phenyl]-3-(5-methyl-1,3,4-oxadiazol-2-yl)benzenesulphonamide

To a solution of 4-dimethyl amino aniline (0.05 g, 0.367 mmol) and triethylamine (0.056 mL, 0.404 mmol, 1.1 eq.) in acetonitrile (2 mL) under nitrogen was added 3-(5-methyl-1,3,4-oxadiazol-2-yl)benzene sulphonyl chloride (0.0997 g, 0.385 mmol, 1.05 eq.) in acetonitrile (2 mL). The mixture was shaken at room temperature for 18 hours. The solvent was removed in vacuo. The residue was re-dissolved in AcOEt and the organic layer washed with saturated aqueous NaHCO₃, separated, dried (Na₂SO₄) and concentrated in vacuo. The residue was analysed by LCMS and was shown to be mainly product (Rₜ 9.97 min; m/z—359.3). The residue was purified by MS-directed prep HPLC to give the sulphonamide as a black solid (5.6 mg).

¹H NMR (300 MHz CDCl₃/d₄ MeOH (2 drops)) δ 8.29-8.27 (m, 1H), 8.04-8.01 (m, 1H), 7.97-7.94 (m, 1H), 7.81-7.75 (m, 1H), 7.52-7.46 (t, 1H), 7.02-6.97 (m, 4H), 2.96 (s, 6H), 2.67 (s, 3H); Purity—>95%.

### EXAMPLE 7

### 2,4-dichloro-N-[4-(2,6,6-trimethyl-4-oxo-4,5,6,7-tetrahydro-1H-indol-1-yl)-phenyl]benzenesulphonamide

To bromo wang resin in DMF (4 ml) was added 1-(4-aminophenyl)-2,6,6-trimethyl-5,6,7-trihydroindol-4-one (0.375 g, 1.40 mmol, 5 eq.), sodium iodide (0.210 g, 1.40 mmol, 5 eq.) and disopropylethylamine (0.500 ml, 2.80 mmol, 10 eq.). The resin was shaken at 90° C. for 24 hrs. The resin was filtered and washed with 5 ml of DMF, DCM, DMF, DCM, MeOH, DCM, MeOH and finally Et₂O. The resin was dried under vacuum.

To the resin was added pyridine (3 ml), 2,4-dichlorobenzene sulphonyl chloride (0.436 g, 1.75 mmol, 5 eq.) and DMAP (0.085 g, 0.700 mmol, 2 eq.). The resin was shaken at 60° C. for 18 hrs and washed with 5 ml of DMF, DCM, DMF, DCM, MeOH, DCM, MeOH and finally Et₂O.

The resin was shaken in a solution of 95% TFA/5% H₂O (3 ml) for 24 hrs, filtered and the resin washed with DCM (1 ml) and MeOH (1 ml). The combined filtrates were concentrated in vacuo. The residue was purified by MS-directed prep HPLC to give the sulphonamide (1.1 mg).

LCMS Rₜ 11.46 min.; M/z—478; Purity—85%.

### EXAMPLE 8

### 2,4-dichloro-N-(2-methyl-1,3-benzothiazol-5-yl)benzenesulphonamide

To a solution of 2-methyl-1,3-benzothiazol-5-amine (0.05 g, 0.211 mmol, 1 eq.) in acetonitrile (2 ml) was added triethyl amine (0.059 ml, 0.232 mmol, 1.1 eq.) and 2,4 dichlorobenzene sulphonyl chloride (0.054 g, 0.222 mmol, 1.05 eq.). The mixture was shaken at room temperature for 18 hours. The solvent was removed in vacuo and the residue dissolved in AcOEt. The AcOEt was washed with saturated aqueous NaHCO₃, separated, dried (Na₂SO₄) and concentrated in vacuo. The residue was purified by MS-directed prep HPLC to yield the sulphonamide (3.1 mg).

LCMS R_{T} 11.15 min.; m/z—374; Purity—95%.

### EXAMPLE 9

### 4-bromo-N-[4-(dimethylamino)phenyl]benzenesulphonamide

To a solution of 4-dimethyl amino aniline (2 g, 0.0147 mol) and triethylamine (2.25 mL, 0.0162 mol, 1.1 eq.) in acetonitrile (20 mL) at 0° C. under nitrogen was added 4-bromo-benzene sulphonyl chloride (3.94 g, 0.0154 mol, 1.05 eq.). The mixture was cooled to 0° C. for 30 mins, and then allowed to warm to room temperature. The reaction was stirred for 18 hours. The solvent was removed in vacuo and the residue redissolved in ethyl acetate (100 mL). The organic layer was washed with sat aqueous NaHCO₃ (2×200 mL), water (2×200 mL), brine (200 mL), separated, dried (Na₂SO₄), filtered and concentrated in vacuo. The residue was dissolved in DCM, filtered through a pad of silica and the pad washed twice with DCM (100 ml). The filtrates were combined and concentrated in vacuo. The sulphonamide was obtained as a orange coloured solid (4.0 g, 76.6%).

¹H NMR (300 MHz CDCl₃) δ 7.47 (s, 4H), 6.83-6.71 (d, 2H), 6.50-6.46 (d, 2H), 6.31 (b s, 1H), 2.83 (s, 6H); ¹³C NMR (300 MHz CDCl₃) δ 149.92, 138.83, 132.47, 129.32, 128.00, 126.77, 124.40, 113.07, 40.86; LCMS Rₜ 11.57 min.; m/z—356:358 (1:1 ratio).

### EXAMPLE 10

### N-[4-(dimethylamino)phenyl]-4-(1-naphthyl)benzenesulfonamide

4-bromo-N-[4-(dimethylamino)phenyl]benzenesulphonamide (25 mg, 0.07 mmol) and 1-naphthyl boronic acid (17.2 mg, 0.07 mmol, 1 eq.) was dissolved in toluene (2 ml) under N₂. Saturated aqueous Na₂CO₃ (1 ml) was added followed by palladium tetrakis(triphenylphosphine) (1 mg, cat.). The reaction was refluxed for 4 hrs and then left to stirring at room temperature for 18 hrs. The reaction was diluted with AcOEt (4 ml) and the organic layer decanted off. The organic layer was filtered through a pad of celite and the solvent removed in vacuo. The residue was analysed by LCMS and confirmed to be the sulphonamide product (17.2 mg, 60.4%).

LCMS Rₜ 12.91 min.; m/z—404; Purity—95%.

The compounds of example 11 to 71 were prepared according to the following general procedures.

### Sulfonyl Chloride Coupling Procedure 1: Coupling of Sulfonyl Chloride to Amine in Acetonitrile.

To a stirred solution of the amine (0.75 mmol) and triethylamine (0.75 mmol) in anhydrous acetonitrile (1 ml) at 0° C. was added 2,4-dichloro-benzenesulphonyl chloride (0.50 mmol) in acetonitrile (1 ml). The mixture was stirred at this temperature for 2-3 hours and/or warmed up to ambient temperature and stirred until reaction had completed by TLC.

The solvent was removed in vacuo and the residue partitioned between ethyl acetate (25 ml) and saturated aqueous sodium bicarbonate solution (25 ml). The organic layer was separated and further washed with sodium bicarbonate (2×25 ml), brine (2×25 ml), dried over sodium sulphate and concentrated down. The product was purified either by flash chromatography (cyclohexane/ethyl acetate eluent on silica), preparative HPLC (acetonitrile/water on C18 silica column), using a silica cartridge (cyclohexane/ethyl acetate eluent on silica), preparative HPLC (either reverse C18 or normal silica) or by recrystallisation from methanol.

Sulfonyl Chloride Coupling Procedure 2: Coupling of Sulfonyl Chloride to amine in pyridine.

To the aniline (0.6 mmol) in pyridine (5 ml) stirring at 0° C. was added sulfonyl chloride (1 equivalent) in pyridine (5 ml) and the reaction was allowed to warm to room temperature overnight. The solvent was evaporated and the resulting residue taken up in EtOAc and washed with aqueous solution of base. The rest of the workup as was for sulfonyl chloride procedure 1.

### Suzuki Coupling Procedure 1

To a degassed mixture of toluene (4 ml) and 2M aqueous Na₂CO₃ (2 ml) was added the bromosulfonamide (0.26 mmol), the phenyl boronic acid (0.28 mmol) and tetrakis(triphenylphosphine) palladium(0) (3 to 5 mol %). The mixture was refluxed for 48 hours. The reaction was cooled, filtered through celite and the celite cake washed with AcOEt (3*50 ml). The organic layer was dried and residue purified.

### Suzuki Coupling Procedure 2

To a degassed solution of 3-bromo-N-[4-(dimethylamino)phenyl]-benzenesulfonamide (100 mg, 0.28 mmol) in toluene (2.5 ml) was added tetrakis(triphenylphosphine) palladium(0) (10 mg, 3 mol %), pyridyl boronic acid (38 mg, 0.28 mmol) in ethanol (1 ml) and sodium carbonate (150 mg, 1.41 mmol) in water (1 ml). The reaction was refluxed for 48 hours. The workup procedure was for Suzuki coupling procedure 1.

### Methylation Procedure 1

To a solution of the indole (1 eqv) in N,N-dimethylformamide solvent (0.7 ml/mmol) was added anhydrous potassium carbonate (0.20 eqv.) and dimethyl carbonate (2.1 eqv.). The mixture was stirred under reflux for 2-3 hours before being left to stir at room temperature overnight. The mixture was cooled (5° C.) and ice-cold water (1.5 ml/mmol) was added slowly. The precipitated product is filtered under suction, washed with water and dried in vacuo to give the corresponding N-methylated indole which was then purified.

### Methylation Procedure 2

The sulfonamide (0.14 mmol) was stirred at 0° C. in DMF (anhydrous, 10 ml) with sodium hydride (1 equivalent) for 30 mins. Methyl iodide (1 equivalent) was added and the reaction allowed to rise to room temperature with stirring. The reaction was monitored by TLC and if necessary further methyl iodide added. The reaction solution was then diluted into distilled water and extracted with ethyl acetate, the ethyl acetate was repeatedly washed with distilled water and then brine before being dried (sodium sulphate) and evaporated to dryness prior to purification.

### Methylation Procedure 3

The sulphonamide (1 eqv) and 1,4-diazabicyclo[2.2.2]octane (0.2 eqv) were heated in DMF/Dimethyl carbonate (1/10 mixture, 10 ml) at 95° C. for 1 to 3 days. The mixture was allowed to cool to room temperature and partitioned between ethyl acetate (15 ml) and water (15 ml). The organic layer was separated and washed with water (10 ml), 10% citric acid (2×10 ml) and again with water (2×10 ml). The organics were dried over sodium sulphate and concentrated in vacuo.

### Reduction of the Nitro Group

To a suspension of the nitroindole (1.0 mmol) and Raney nickel (36.5 mg) in ethanol (7.5 ml) was added hydrazine hydrate (365 □l) drop-wise. The mixture was heated under reflux for 25 minutes, then cooled and filtered and evaporated to give the aminoindole in a pure enough state to carry out subsequent reactions.

### EXAMPLE 11

### Compound 131 2,4-dichloro-N-(2-methyl-1H-indol-5-yl)-benzenesulfonamide

Synthesised according to general coupling procedure 1 and purified by flash chromatography.

¹H NMR (300 MHz; CDCl₃) 2.38 (3H, s, 2′-CH₃), 6.11 (1H, m, 3′-H), 6.84 (1H, dd, J 2.1 and 8.5 Hz), 6.96 (1H, s), 7.09 (1H, d, J 8.5 Hz), 7.16 (1H, dd, J 2.0 and 8.5 Hz), 7.23 (1H, d, J 2.0 Hz), 7.50 (1H, d, J 2.0 Hz), 7.77 (1H, d, J 8.5 Hz), 7.93 (1H, br, N—H)

¹³C NMR (300 MHz, CDCl₃) 13.69 (CH₃), 100.66 (3′-CH), 110.75 (CH), 115.44 (CH), 117.83 (CH), 127.14, 127.45 (CH), 129.35, 131.17 (CH), 132.24, 132.99 (CH), 134.74, 135.07, 136.80, 139.51

Actual Mass: 354.95

LCMS: Mass detected [M−H]⁻ 353.00; Retention time 17.2 mins; Purity 87%

### EXAMPLE 12

### Compound 132 2,4-dichloro-N-(2-methyl-benzothiazol-5-yl)-benzenesulfonamide

Synthesised according to general coupling procedure 1 and purified by flash chromatography.

¹H NMR (300 MHz; CDCl₃) 2.79 (3H, s, 2′-CH₃), 7.18-7.28 (3H, m, 3×Ar—H), 7.49 (1H, d, J 2.0 Hz), 7.67 (1H, d, J 8.6 Hz), 7.94 (1H, d, J 11.5 Hz)

¹³C NMR (300 MHz, CDCl₃) 20.19 (CH₃), 115.59 (CH), 119.82 (CH), 122.13 (CH), 127.65 (CH), 131.46 (CH), 132.26, 133.04 (CH), 133.44, 134.61, 140.08, 153.89, 169.12

Actual Mass: 404.85

LCMS: Mass detected [M−H]-402.85; Retention time 16.6 mins; Purity 96%

### EXAMPLE 13

### Compound 133 2,4-dichloro-N-(2-methyl-benzothiazol-6-yl)-benzene-sulfonamide

Synthesised according to general coupling procedure 1 and purified by flash chromatography.

¹H NMR (300 MHz; CDCl₃) 2.78 (3H, s, 2′-CH₃), 7.12 (1H, dd, J 2.2 and 8.7 Hz), 7.15 (1H, br, N—H), 7.27 (1H, dd, J 2.0 and 8.5 Hz), 7.52 (1H, d, J 2.0 Hz), 7.67 (1H, d, J 2.2 Hz), 7.76 (1H, d, J 8.7 Hz), 7.88 (1H, d, J 8.5 Hz)

Actual Mass: 373.00

LCMS: Mass detected [M−H]-370.95; Retention time 13.2 mins; Purity 97%

### EXAMPLE 14

### Compound 134 2,4-dichloro-N-(1H-indol-5-yl)-benzenesulfonamide

Synthesised according to general coupling procedure 1 and purified by flash chromatography.

¹H NMR (300 MHz; CDCl₃) 2.04 (3H, s, 2′-CH₃), 6.45-6.46 (1H, m), 6.94 (1H, dd, J 2.0 and 8.6 Hz), 7.00 (1H, s), 7.17-7.25 (3H, m), 7.38 (1H, d, J 1.6 Hz), 7.52 (1H, d, J 2.0 Hz), 7.80 (1H, d, J 8.5 Hz), 8.25 (1H, br, N—H)

Actual Mass: 341.00

LCMS: Mass detected [M−H]-339.05; Retention time 13.2 mins; Purity 96%

### EXAMPLE 15

### Compound 138 2,4-dichloro-N-benzothiazol-6-yl)-benzenesulfonamide

Synthesised according to general coupling procedure 1 and purified by flash chromatography.

¹H NMR (300 MHz; CDCl₃) 7.22 (1H, dd, J 2.2 and 8.7 Hz), 7.28 (1H, dd, J 2.0 and 8.5 Hz), 7.51 (1H, d, J 2.0 Hz), 7.56 (1H, br, N—H), 7.82 (1H, d, J 2.1 Hz), 7.94 (2H, dd, J 8.7 and 13.3 Hz), 8.94 (1H, s, 2′-H)

Actual Mass: 358.90

LCMS: Mass detected [M−H]⁻ 356.90; Retention time 12.2 mins; Purity 88%

### EXAMPLE 16

### Compound 139 N-benzolthiazol-6-yl-3-bromo-benzenesulfonamide

Synthesised according to general coupling procedure 1 and purified by flash chromatography.

NMR—To be purified and determined.

Actual Mass: 369

LCMS: No ionization; Retention time 10.3 mins; Purity 93%

### EXAMPLE 17

### Compound 140 3-bromo-N-(2-methyl-benzothiazol-5-yl)-benzenesulfonamide

Synthesised according to general coupling procedure 1 and purified by flash chromatography.

¹H NMR (300 MHz; CDCl₃) 2.80 (3H, s, 2′-CH₃), 7.18 (1H, dd, J 2.1 and 8.6 Hz), 7.26 (1H, dd, J 2.7 and 10.6 Hz), 7.33 (1H, br, N—H), 7.60-7.72 (4H, m, 4×Ar—H), 7.94 (1H, m, Ar—H)

Actual Mass: 383

LCMS: No ionization; Retention time 17.1 mins; Purity 93%

### EXAMPLE 18

### Compound 156 2,4-dichloro-N-(2-methyl-benzooxazol-5-yl)-benzene-sulfonamide

Synthesised according to general coupling procedure 1 and purified by flash chromatography.

¹H NMR (300 MHz; CDCl₃) 2.59 (3H, s, 2′-CH₃), 7.07 (1H, br, N—H), 7.11 (1H, dd, J 2.2 and 8.6 Hz), 7.24 (1H, dd, J 2.0 and 8.6 Hz), 7.34 (1H, d, J 8.6 Hz), 7.38 (1H, d, J 2.0 Hz), 7.52 (1H, d, J 2.0 Hz), 7.84 (1H, d, J 8.6 Hz)

Actual Mass: 356.80

LCMS: Mass detected [M−H]⁻ 355.00; Retention time 17.6 mins; Purity 80%

### EXAMPLE 19

### Compound 157 N-benzo[1,3]dioxol-5-yl-2,4-dichloro-benzenesulfonamide

Synthesised according to general coupling procedure 1 and purified by flash chromatography.

¹H NMR (300 MHz; CDCl₃) 5.92 (2H, s, 2′-CH₂), 6.50 (1H, dd, J 2.1 and 8.3 Hz), 6.61 (1H, d, J 8.3 Hz), 6.70 (1H, d, J 2.1 Hz), 6.93 (1H, br, N—H), 7.30 (1H, dd, J 2.2 and 8.5 Hz), 7.53 (1H, d, J 2.0 Hz), 7.86 (1H, d, 8.5 Hz)

Actual Mass: 345.95

LCMS: Mass detected [M−H]⁻ 343.80; Retention time 14.3 mins; Purity 97%

### EXAMPLE 20

### Compound 158 3-bromo-N-(2-methyl-benzooxazol-5-yl)-benzenesulfonamide

Synthesised according to general coupling procedure 1 and purified by flash chromatography.

¹H NMR (300 MHz; CDCl₃) 2.62 (3H, s, 2′-CH₃), 6.70 (1H, br, N—H), 7.06 (1H, dd, J 2.2 and 8.6 Hz), 7.25-7.31 (2H, m 2×Ar—H), 7.37 (1H, d, J=8.6 Hz), 7.59-7.64 (2H, m, 2×Ar—H), 7.90 (1H, t, J 1.8 Hz)

Actual Mass: 367.00

LCMS: Mass detected [M−H]-365.00; Retention time 11.1 mins; Purity 86%

### EXAMPLE 21

### Compound 159 N-benzo[1,3]dioxol-5-yl-3-bromo-benzenesulfonamide

Synthesised according to general coupling procedure 1 and purified by flash chromatography.

¹H NMR (300 MHz; CDCl₃) 5.95 (2H, s, 2′-CH₂), 6.44 (1H, dd, J 2.2 and 8.3 Hz), 6.65 (1H, d, J 8.3 Hz), 6.67 (1H, d, J 2.2 Hz), 6.80 (1H, br, N—H), 7.32 (1H, t, J 7.9 Hz), 7.65 (2H, dt, J 0.9 and 7.9 Hz), 7.90 (1H, t, J 1.8 Hz)

Actual Mass: 356.00

LCMS: Mass detected [M−H]-353.95; Retention time 13.4 mins; Purity 98%

### EXAMPLE 22

### Compound 160 2,4-dichloro-N-(2-methyl-benzooxazol-6-yl)-benzenesulfonamide

Synthesised according to general coupling procedure 1 and purified by flash chromatography.

¹H NMR (300 MHz; CDCl₃) 2.59 (3H, s, 2′-CH₃), 7.00 (1H, dd, J 3.1 and 6.4 Hz), 7.26 (1H, dd, J 2.0 and 8.5 Hz), 7.39 (1H, d, J 2.0 Hz), 7.43 (1H, d, J 4.7 Hz), 7.49 (1H, d, J 2.0 Hz), 7.67 (1H, br, N—H), 8.17 (1H, d, J 8.5 Hz)

Actual Mass: 357.00

LCMS: Mass detected [M−H]-355.00; Retention time 11.7 mins; Purity 99%

### EXAMPLE 23

### Compound 169 3-bromo-N-(2-methyl-benzooxazol-6-yl)-benzenesulfonamide

Synthesised according to general coupling procedure 1 and purified by flash chromatography.

¹H NMR (300 MHz; CDCl₃) 2.62 (3H, s, 2′-CH₃), 6.91 (1H, dd, J 2.0 and 8.4 Hz), 7.28 (1H, t, J 7.9 Hz), 7.38-7.40 (2H, m), 7.47 (1H, d, J 8.5 Hz), 7.61-7.66 (2H, m), 7.9 (1H, t, J 1.8 Hz)

Actual Mass: 366.95

LCMS: Mass detected [M−H]-364.90; Retention time 10.6 mins; Purity 89%

### EXAMPLE 24

### Compound 161 2,4-dichloro-N-(1H-indol-6-yl)-benzenesulfonamide

Synthesised according to general coupling procedure 1 and purified by flash chromatography.

¹H NMR (300 MHz; CDCl₃) 6.47 (1H, m), 6.76 (1H, dd, J 1.9 and 8.4 Hz), 7.50 (1H, s), 7.18-7.26 (3H, m), 7.32 (1H, s), 7.44 (1H, d, J 8.4 Hz), 7.51 (1H, d, J 2.0 Hz), 7.82 (1H, d, J 8.5 Hz), 8.21 (1H, br, N—H)

Actual Mass: 341.05

LCMS: Mass detected [M−H]-339.05; Retention time 14.1 mins; Purity 99%

### EXAMPLE 25

### Compound 162 3-bromo-N-(1H-indol-6-yl)-benzenesulfonamide

Synthesised according to general coupling procedure 1 and purified by flash chromatography.

¹H NMR (300 MHz; CDCl₃) 6.51 (1H, m), 6.57 (1H, s), 6.64 (1H, dd, J 1.9 and 8.4 Hz), 7.22 (1H, dd, J 2.4 and 5.6 Hz), 7.33 (1H, s), 7.47 (1H, d, J 8.4 Hz), 7.55-7.62 (2H, m), 7.91 (1H, t, 1.8 Hz), 8.22 (1H, br, N—H)

Actual Mass: 350.90

LCMS: Mass detected [M−H]⁻ 348.90; Retention time 12.9 mins; Purity 98%

### EXAMPLE 26

### Compound 130 4-bromo-2-chloro-N-(4-dimethylamino-phenyl)-benzenesulfonamide

Synthesised according to general coupling procedure 1 and purified by flash chromatography.

¹H NMR 300 MHz; δ_{H} (CDCl₃) 7.73 (1H, d, J 8.4 Hz, ArH), 7.69 (1H, d, J 2.0 Hz, ArH), 7.41 (1H, dd, J 2.0, 8.4 Hz, ArH), 6.97 (2H, d, J 8.8 Hz, ArH), 6.54 (2H, d, J 8.8 Hz, ArH), 2.90 (6H, s, N(CH₃)₂).

ESMS+ve calculated 389.7, [M+H]⁺ 389.17. Purity Estimated >90%

### EXAMPLE 27

### Compound 141 4-bromo-N-(2,4-dichloro-phenyl)-benzenesulfonamide

Synthesised according to general coupling procedure 1 and purified by flash chromatography.

¹H NMR 400 MHz δ_{H} (DMSO) 7.93 (4H, m, ArH), 7.75 (2H, dd, J 2.0, 7.2 Hz), 7.32 (1H, J 7.2 Hz, ArH).

Actual Mass: 381.08

LCMS: Mass detected [M−H]⁻ no ionisation; Retention time 16.25 mins; Purity 95.2%

### EXAMPLE 28

### Compound 167 4-bromo-N-(3,4-dichloro-phenyl)-benzenesulfonamide

Synthesised according to general coupling procedure 1 and purified by flash chromatography.

¹H NMR 400 MHz δ_{H} (DMSO) 7.92 (2H, d, J 8.8 Hz, ArH), 7.67 (2H, d, J 8.8 Hz, ArH). 7.66 (1H, d, ArH), 7.50 (1H, d, J 2.0 Hz, ArH), 7.04 (1H, dd, J 2.0, 7.6 Hz, ArH).

Actual Mass: 381.08

LCMS: Mass detected [M−H]⁻ 380.10; Retention time 21.57 mins; Purity 92.1%

### EXAMPLE 29

### Compound 135 [4-(2,4-dichloro-benzenesulfonylamino)-phenyl]-(4,6-dimethyl-pyrimidin-2-yl)-methyl-ammonium; chloride

Compound 50 2,4-dichloro-N-{4-[(4,6-dimethyl-pyrimidin-2-yl)-methyl-amino]-phenyl}-benzenesulfonamide (75 mg, 1.7 mM) was dissolved in ethyl acetate (10 ml) with stirring. To this solution was carefully added a solution of 2M hydrochloric acid in diethyl ether (1 ml). A white precipitate is then observed. This solid was filtered off, washed with diethyl ether and dried under high vacuum. The salt produced was redissolved in distilled water with a minimum of acetonitrile to ensure complete solubility and freeze dried to yield an off white solid.

¹H NMR 300 MHz δ_{H} (CD₃OD) 9.37 (1H, d, J 8.4 Hz, ArH), 8.98 (1H, d, J 2.0 Hz, ArH), 8.83 (1H, dd, J 2.0, 8.4 Hz, ArH), 8.57 (4H, m, ArH), 7.94 (1H, s, Pyrimidyl), 3.50 (6H, ArCH₃).

Purity Estimated >90%

### EXAMPLE 30

### Compound 136 Methanesulfonate[4-(2,4-dichloro-benzenesulfonylamino)-phenyl]-(4,6-dimethyl-pyrimidin-2-yl)-methyl-ammonium

Compound 50 2,4-dichloro-N-{4-[(4,6-dimethyl-pyrimidin-2-yl)-methyl-amino]-phenyl}-benzenesulfonamide (75 mh, 1.7 mM) was dissolved in ethyl acetate (10 ml) with stirring. To this solution is added a solution of methane sulfonic acid in ethyl acetate (1M, 2 ml), this solution was then evacuated to dryness to yield a light brown oil. The oil was repeatedly suspended in dry diethyl ether and the solvent decanted off to remove excess acid. The salt produced was the redissolved in distilled water with a minimum of acetonitrile to ensure complete solubility and freeze dried to yield a brown oil.

¹H NMR 300 MHz δ_{H} (CD₃OD) 9.46 (1H, d, J 8.4 Hz, ArH), 9.01 (1H, d, J 2.0 Hz, ArH), 8.88 (1H, dd, J 2.0, 8.4 Hz, ArH), 8.74 (2H, d, ArH), 8.66 (2H, d, ArH), 8.24 (1H, s, Pyrimidyl), 4.83 (3H, s), 3.75 (6H, ArCH₃).

Purity Estimated >90%

### EXAMPLE 31

### Compound 142 [4-(3′,4′-dimethoxy-biphenyl-3-sulfonylamino)-phenyl]-dimethylammonium; chloride

Procedure used identical to that for compound 135 using compound 102 as the starting material.

¹H NMR 400 MHz δ_{H} (CDCl₃) 7.94 (1H, s, ArH), 7.89 (1H, d, J 7.6 Hz, ArH), 7.65 (1H, d, J 7.6 Hz, ArH), 7.58 (1H, t, J 7.6 Hz, ArH), 7.05-7.16 (7H, m, ArH), 3.83 (3H, s, OCH₃), 3.79 (3H, s, OCH₃), 2.92 (6H, s, N(CH₃)₂)

Purity Estimated >90%

### EXAMPLE 32

### Compound 137 4-bromo-2-chloro-N-{4-[(4,6-dimethyl-pyrimidin-2-yl)-methylamino]-phenyl}-benzenesulfonamide

Synthesised according to general coupling procedure 1 and purified by flash chromatography

¹H NMR 400 MHz δ_{H} (CDCl₃) 7.78 (1H, d, J 8.8 Hz, ArH), 7.69 (1H, s), 7.48 (1H, d, J 8.8 Hz, ArH), 7.25 (2H, d, J 8.7 Hz, ArH), 7.06 (2H, d, J 8.7 Hz, ArH), 6.37 (1H, s, Pyrimidyl), 3.48 (3H, s), 2.25 (6H, s).

Actual Mass: 481.80

LCMS: Mass detected [M−H]-481.30; Retention time 16.58 mins; Purity 96.6%

### EXAMPLE 33

### Compound 164 2,4-dichloro-N-[4-(4,6-dimethoxy-pyrimidin-2-yl)-phenyl]-benzenesulfonamide

Synthesised according to general coupling procedure 1 and purified by flash chromatography.

¹H NMR 300 MHz δ_{H} (CDCl₃) 8.6 (2H, d, J 7.6 Hz, ArH), 7.98 (1H, d, J 8.5 Hz, ArH), 7.49 (1H, d, J 2.0 Hz, ArH), 7.30 (1H, dd, J 2.0, 8.5 Hz, ArH), 7.18 (2H, d, J 7.5 Hz, ArH), 7.14 (1H, br s, NH), 5.93 (1H, s, Pyrimidyl), 4.00 (6H, s, OCH₃).

Actual Mass: 440.30

LCMS: Mass detected [M−H]⁻ No Ionisation; Retention time 16.04 mins; Purity 96.9%

### EXAMPLE 34

### Compound 165 2,4-dichloro-N-[4-(4,6-dimethyl-pyrimidin-2-yloxy)-phenyl]-benzenesulfonamide

Synthesised according to general coupling procedure 1 and purified by flash chromatography.

¹H NMR 300 MHz δ_{H} (CDCl₃) 7.91 (1H, d, J 8.5 Hz, ArH), 7.54 (1H, d, J 2.0 Hz, ArH), 7.31 (1H, dd, J 2.0, 8.5 Hz, ArH), 7.12 (4H, m, AB d), 6.96 (1H, br s, NH), 6.76 (1H, s, pyrimidyl), 2.37 (6H, s).

Actual Mass: 424.31

LCMS: Mass detected [M−H]-422.40; Retention time 13.59 mins; Purity 97.0%

### EXAMPLE 35

### Compound 168 2,4-dichloro-N-[4-(4,6-dimethyl-pyrimidin-2-ylsulfonyl)-phenyl]-benzenesulfonamide

Synthesised according to general coupling procedure 1 and purified by flash chromatography.

¹H NMR 400 MHz δ_{H} (CDCl₃) 7.95 (1H, d, J 8.5 Hz, ArH), 7.49 (1H, d, J 2.0 Hz, ArH), 7.45 (1H, d, J 8.4 Hz, ArH), 7.30 (1H, dd, J 2.0, 8.4 Hz, ArH), 7.11 (2H, d, J 8.4 Hz, ArH), 6.67 (1H, br s, NH), 2.27 (6H, s, CH₃)

Actual Mass: 440.37

LCMS: Mass detected [M−H]-438.40; Retention time 16.25 mins; Purity >95%

### EXAMPLE 36

### Compound 163 2,4-dichloro-N-(4-pyrrol-1-yl-phenyl)-benzenesulfonamide

Synthesised according to general coupling procedure 1 and purified by prep HPLC.

¹H NMR 300 MHz δ_{H} (CDCl₃) 7.90 (1H, d, J 8.4 Hz, ArH), 7.54 (1H, d, J 2.0 Hz, ArH), 7.30 (1H, dd, J 2.0, 8.4 Hz, ArH), 7.25 (2H, d, ArH), 7.17 (2H, d, ArH), 6.98 (2H, t, J 2.0 Hz, Pyrrole), 6.31 ((2H, t, J 2.0 Hz, Pyrrole).

Actual Mass: 367.27

LCMS: Mass detected [M−H]⁻ 365.20; Retention time 16.55 mins; Purity 96.8%

### EXAMPLE 37

### Compound 166 Biphenyl-3-sulfonic acid (4-dimethylamino-phenyl)-amide

Synthesised according to general coupling procedure 1 and purified by Prep HPLC.

¹H NMR 400 MHz δ_{H} (CDCl₃) 7.82 (1H, t, ArH), 7.73 (1H, td, J 7.8 Hz, ArH), 7.64 (1H, td, J 7.8 Hz, ArH), 7.33-7.49 (6H, m, ArH), 6.90 (2H, d, J 8.8 Hz, ArH), 6.56 (2H, d, ArH), 6.12 (1H, br s, ArH), 2.90 (6H, s, N(CH₃)₂.

Actual Mass: 352.46

LCMS: Mass detected [M−H]⁻ 351.4; Retention time mins; Purity 98.5%

### EXAMPLE 38

### Compound 262 2′,6′-dimethoxy-biphenyl-3-sulfonic acid (4-dimethylaminophenyl)-amide

Synthesised according to Suzuki coupling procedure 1. Purification by prep HPLC provided (compound 262) (6.9 mg) as a solid.

¹H NMR (300 MHz, CDCl₃) δ7.78 (s, 1H), 7.57-7.25 (m, 4H), 6.96-6.92 (m, 2H), 6.64-6.56 (m, 4H), 6.15 (br s, 1H), 3.69 (s, 6H), 2.92 (s, 6H); LCMS Rₜ 14.36 min.; purity 91%; MS m/z 413.3 [M+H]⁺.

### EXAMPLE 39

### Compound 197 3-bromo-N-(2-methyl-1H-indol-5-yl)-benzenesulfonamide

Synthesised according to sulfonyl chloride coupling procedure 2a and purified by flash chromatography. Yield: 77%

¹H NMR (300 MHz; CDCl₃) δ8.13 (br, 1H), 7.89 (m, 1H), 7.63-7.53 (m, 2H), 7.26-7.19 (m, 3H), 7.13 (d, 1H, J=8.5 Hz), 6.76 (dd, 1H, J=2.1 and 8.5 Hz), 6.49 (s, 1H), 6.14 (m, 1H), 2.42 (s, 3H).

LCMS Rt 8.38 min.; purity 96.7%; MS m/z 363 [M−H]⁻

### EXAMPLE 40

### Compound 184 4′-fluoro-biphenyl-3-sulfonic acid (1H-indol-5-yl)-amide

5-aminoindole was coupled to 3-bromobenzene sulfonyl chloride according to sulfonyl coupling procedure 1 and reacted with 4-fluoroboronic acid as described in Suzuki coupling procedure 1. The final product was purified by HPLC. Yield 78%

¹H NMR (300 MHz; CD₃OD) δ 7.71-7.62 (m, 3H), 7.53-7.47 (m, 1H), 7.32-7.21 (m, 4H), 7.08-7.02 (m, 2H), 6.83 (dd, 1H, J=2.1 and 8.6 Hz), 6.34 (dd, 1H, J=0.6 and 3.1 Hz).

LCMS Rt 18.88 min.; purity 78.5%; MS m/z 365 [M−H]⁻

### EXAMPLE 41

Compound 185 4′-fluoro-biphenyl-3-sulfonic acid (1H-indol-6-yl)-amide

Compound 262 was reacted with 4-fluoroboronic acid as described in Suzuki coupling procedure 1. The final product was purified by HPLC. Yield 57%

¹H NMR (300 MHz; CD₃OD) δ 7.73-7.67 (m, 3H), 7.51-7.47 (m, 1H), 7.39-7.29 (m, 3H), 7.21-7.19 (m, 2H), 7.11-7.07 (m, 2H), 6.69 (dd, 1H, J=1.9 and 8.6 Hz), 6.38 (dd, 1H, J=0.9 and 3.2 Hz).

LCMS Rt 19.35 min.; purity 78.0%; MS m/z 365 [M−H]⁻

### EXAMPLE 42

### Compound 186 4′-fluoro-biphenyl-3-sulfonic acid benzo[1,3]dioxol-5-yl-amide

Compound 159 was reacted with 4-fluoroboronic acid as described in Suzuki coupling procedure 1. The final product was purified by HPLC. Yield 65%

¹H NMR (300 MHz; CD₃OD) δ 7.92-7.79 (m, 2H), 7.70-7.68 (m, 1H), 7.58-7.53 (m, 3H), 7.22-7.16 (m, 2H), 6.66-6.63 (m, 2H), 6.47 (dd, 1H, J=2.1 and 8.3 Hz), 5.88 (s, 2H).

LCMS Rt 15.39 min.; purity 77.0%; MS m/z 370 [M−H]⁻

### EXAMPLE 43

### Compound 187 4′-fluoro-biphenyl-3-sulfonic acid (2-methyl-benzooxazol-6-yl)-amide

Compound 169 was reacted with 4-fluoroboronic acid as described in Suzuki coupling procedure 1. The final product was purified by HPLC. Yield 69%

¹H NMR (300 MHz; CD₃OD) δ 7.89-7.87 (m, 1H), 7.80-7.78 (m, 1H), 7.73-7.69 (m, 1H), 7.56-7.49 (m, 3H), 7.42 (d, 1H, J=8.5 Hz), 7.38 (m, 1H), 7.18-7.13 (m, 2H), 7.02 (dd, 1H, J=2.0 and 8.5 Hz), 2.56 (s, 3H).

LCMS Rt 14.16 min.; purity 71.9%; MS m/z 381 [M−H]⁻

### EXAMPLE 44

### Compound 188 4′-fluoro-biphenyl-3-sulfonic acid (2-methyl-benzothiazol-5-yl)-amide

Compound 140 was reacted with 4-fluoroboronic acid as described in Suzuki coupling procedure 1. The final product was purified by HPLC. Yield 76%

¹H NMR (300 MHz; CD₃OD) δ 7.88 (m, 1H), 7.78-7.71 (m, 3H), 7.62 (d, 1H, 1.3 Hz), 7.55-7.46 (m, 3H), 7.22-7.12 (m, 3H), 2.76 (s, 3H)

LCMS Rt 19.78 min.; purity 69.0%; MS m/z 397 [M−H]⁻

### EXAMPLE 45

### Compound 189 4′-fluoro-biphenyl-3-sulfonic acid benzothiazol-6-ylamide

Compound 139 was reacted with 4-fluoroboronic acid as described in Suzuki coupling procedure 1. The final product was purified by HPLC. Yield 50%

¹H NMR (300 MHz; CD₃OD) δ 9.14 (s, 1H), 7.93-7.85 (m, 3H), 7.79-7.72 (m, 2H), 7.55-7.45 (m, 3H), 7.27 (dd, 1H, J=2.2 and 8.8 Hz), 7.16-7.11 (m, 2H).

LCMS Rt 14.15 min.; purity 54.0%; MS m/z 383 [M−H]⁻

### EXAMPLE 46

### Compound 190 4′-fluoro-biphenyl-3-sulfonic acid (2-methyl-benzooxazol-5-yl)-amide

Compound 158 was reacted with 4-fluoroboronic acid as described in Suzuki coupling procedure 1. The final product was purified by HPLC. Yield 87%

¹H NMR (300 MHz; CDCl₃) δ 7.90 (s, 1H), 7.67 (dd, 2H, J=1.8 and 7.7 Hz), 7.49-7.32 (m, 5H), 7.15-7.09 (m, 3H), 6.98 (br, 1H), 2.37 (s, 3H)

LCMS Rt 14.46 min.; purity 69.6%; MS m/z 381 [M−H]

### EXAMPLE 47

### Compound 191 4′-fluoro-biphenyl-3-sulfonic acid (2-methyl-1H-indol-6-yl)-amide

Compound 197 was reacted with 4-fluoroboronic acid as described in Suzuki coupling procedure 1. The final product was purified by HPLC. Yield 57%

¹H NMR (300 MHz; (CD₃)₂C═O) δ 8.64 (s, 1H), 7.82-7.79 (m, 2H), 7.69-7.67 (m, 1H), 7.57-7.49 (m, 4H), 7.25 (m, 1H), 7.23-7.17 (m, 4H), 6.88-6.86 (m, 1H), 6.06 (m, 1H), 2.37 (s, 3H)

LCMS Rt 15.47 min.; purity 63.8%; MS m/z 379 [M]⁻

### EXAMPLE 48

### Compound 192 5-bromo-N-[4-(dimethylamino)phenyl]-2,4-difluoro benzene sulfonamide

To a solution of N,N-dimethylbenzene-1,4-diamine dihydrochloride (500 mg, 2.39 mmol) and triethyl amine (1.0 ml, 7.17 mmol) in acetonitrile (30 ml), at 0° C. under N₂, was added dropwise a solution of 5-bromo-2,4-difluorobenzene sulfonyl chloride (697 mg, 2.39 mmol) in acetonitrile (10 ml). The mixture was stirred for 30 minutes and allowed to warm overnight. The solvent was removed in vacuo; the residue redissolved in AcOEt (50 ml) and washed with saturated aqueous NaHCO₃, water, brine, dried (Na₂SO₄) and concentrated in vacuo. The residue was purified by flash column chromatography [AcOEt:cy Hex (2:8)] to yield a mustard coloured solid (547.3 mg).

### EXAMPLE 49

### Compound 219 N-[4-(dimethylamino)phenyl]-3-pyridin-4-ylbenzenesulfonamide

Synthesised according to Suzuki coupling procedure 2 from the respective bromosulfonamides and boronic acid. Purification by flash column chromatography (AcOEt).

¹H NMR (300 MHz CDCl₃+CD₃OD) δ8.51 (d, 2H), 7.79 (s, 1H), 7.72-7.68 (m, 2H), 7.51-7.26 (m, 4H), 6.85 (d, 2H, J=6.84 Hz), 6.52 (d, 2H, J=6.86 Hz), 2.81 (s, 6H).

LCMS Rₜ 11.67 min.; purity 96.3%; MS m/z 354.3 [M+H]⁺.

### EXAMPLE 50

### Compound 220 N-[4-(dimethylamino)phenyl]-3-pyridin-3-ylbenzenesulfonamide

Synthesised according to Suzuki coupling procedure 2 from the respective bromosulfonamides and boronic acid. Purification by flash column chromatography (AcOEt).

¹H NMR (300 MHz, CDCl₃) δ 8.73 (br s, 1H), 8.61 (d, 1H, J=3.76 Hz), 7.85-7.83 (m, 1H), 7.74-7.69 (m, 3H), 7.51 (t, 1H, J=7.79 Hz), 7.37-7.30 (m, 2H), 6.95 (d, 2H, J=6.84 Hz), 6.57 (d, 2H, J=6.87 Hz), 2.89 (s, 6H).

LCMS Rₜ 11.67 min.; purity 96.3%; MS m/z 354.3 [M+H]⁺.

### EXAMPLE 51

### Compound 221 3-({[4-(dimethylamino)phenyl]amino}sulfonyl)-N-[4-(dimethylamino)phenyl]benzamide

To N,N-dimethylbenzene-1,4-diamine dihydrochloride (100 mg, 0.48 mmol) was added 3-(chlorosulfonyl)benzoic acid (106 mg, 0.48 mmol), pyridine (154 μl, 1.91 mmol) and dichloromethane (5 ml). The reaction was stirred for 18 hours at room temperature, diluted with DCM (20 ml), washed twice with 1M aqueous NaHCO₃, dried and concentrated in vacuo. The residue was purified by cartridge column chromatography (AcOEt) to yield a brown solid.

¹H NMR (300 MHz, CDCl₃) δ 8.14 (br s, 1H), 8.07 (d, 1H, J=7.86 Hz), 7.98 (br s, 1H), 7.71 (d, 1H, J=7.92 Hz), 7.48-7.42 (m, 3H), 6.95 (s, 1H), 6.87 (d, 2H, J=6.92 Hz), 6.68 (d, 2H, J=9.04 Hz), 6.52 (d, 2H, J=9.06 Hz), 2.92 (s, 6H), 2.87 (s, 6H).

LCMS Rₜ 13.19 min.; purity 95.7%; MS m/z 439.4 [M+H]⁺.

### EXAMPLE 52

### Compound 222 N-[4-(dimethylamino)phenyl]-4′-fluoro-2′-methyl-1,1′-biphenyl-3-sulfonamide

Synthesised according to Suzuki coupling procedure 1 from the respective bromosulfonamides and boronic acid. Purification by prep HPLC.

¹H NMR (300 MHz, CDCl₃) δ 7.72-7.70 (m, 1H), 7.54-7.44 (m, 3H), 7.02-6.89 (m, 5H), 6.57 (d, 2H, J=7.89 Hz), 6.23 (br s, 1H), 2.91 (s, 6H), 2.08 (s, 3H).

LCMS Rₜ 15.27 min.; purity 94.4%; MS m/z 385.2 [M+H]⁺.

### EXAMPLE 53

### Compound 223 2,4-dichloro-N-[4-(dimethylamino)phenyl]-N-methyl benzenesulfonamide

This product was obtained using methylation procedure 1 from 2,4-Dichloro-N-[4-(dimethylamino)phenyl]benzenesulfonamide The product was purified by preparative layer chromatography [cyclohexane/EtOAc (7:3)].

¹H NMR (300 MHz, CDCl₃) δ 7.74 (d, 1H, J=8.5 Hz), 7.52 (d, 1H, J=2.0 Hz), 7.22 (dd, 1H, J=8.6 Hz, 2.1 Hz) 7.00 (d, 2H, J=9.1 Hz), 6.56 (d, 2H, J=8.9 Hz), 3.38 (s, 3H), 2.92 (s, 6H).

LCMS Rₜ 15.82 min., purity 97%, m/z=359.2.

### EXAMPLE 54

### Intermediate in the Synthesis of compound 223

### Compound 26 2,4-dichloro-N-[4-(dimethylamino)phenyl]benzenesulfonamide

Synthesised according to sulfonyl chloride procedure 1. The crude residue was partitioned between dichloromethane and water, the organic fraction collected and the product purified by flash column chromatography [cyclohexane/EtOAc (8:2-7:3)].

¹H NMR (300 MHz, CDCl₃) δ 7.91 (d, 1H, J=8.4 Hz), 7.54-7.51 (m, 1H), 7.27-7.24 (m, 1H), 6.95 (d, 2H, J=9.0 Hz), 6.74 (s, 1H), 6.53 (d, 2H, J=8.8 Hz), 2.88 (s, 6H).

LCMS [3-97—10 mins] Rₜ 10.2 min., m/z=345.3.

### EXAMPLE 55

### Compound 224 2,4-dichloro-N-(4-isopropylphenyl)benzenesulfonamide

Synthesised ac cording to sulfonyl chloride coupling procedure 1 from the respective sulfonyl chloride and primary amine. The crude residue was purified by flash silica column chromatography [cyclohexane/EtOAc (24:1-47:3)].

¹H NMR (300 MHz, CDCl₃) δ 7.91 (d, 1H, J=8.5 Hz), 7.29 (dd, 1H, J=8.5 Hz, 2 Hz), 7.09-6.98 (m, 5H), 2.81 (septet, 1H, J=6.9 Hz), 1.116 (d, 6H, J=6.9 Hz).

LCMS Rₜ 15.85 min., purity 92%, m/z=no ionisation

### EXAMPLE 56

### Compound 225 3-bromo-N-(4-isopropyl-phenyl)-benzenesulfonamide

Synthesised ac cording to sulfonyl chloride coupling procedure 1 from the respective sulfonyl chloride and primary amine. The crude residue was purified by flash silica column chromatography [cyclohexane/EtOAc (24:1-47:3)].

¹H NMR (300 MHz, CDCl₃) δ 7.87 (t, 1H, J=1.8 Hz), 7.66 (dd, 2H, J=7.9 Hz, 1.8 Hz), 7.31 (t, 1H, J=8.1 Hz), 7.12 (d, 2H, J=8.4 Hz), 6.97 (d, 2H, J=8.5 Hz), 6.55 (s, 1H), 2.85 (septet, 1H, J=6.9 Hz), 1.20 (d, 6H, J=6.9 Hz).

LCMS Rₜ 15.55 min, purity 96%, m/z=no ionisation

### EXAMPLE 57

### Compound 226 N-[4-(1H-imidazol-1-yl)phenyl]naphthalene-2-sulfonamide

Synthesised ac cording to sulfonyl chloride coupling procedure 1 from the respective sulfonyl chloride and primary amine. On taking the crude material up in CH₂Cl₂ to purify a yellow solid precipitated which, on investigation was shown to be pure product.

¹H NMR (300 MHz, CDCl₃) δ 8.37 (d, 1H, J=1.5 Hz), 7.97-7.89 (m, 4H), 7.78 (dd, 1H, J=8.7 Hz, 1.9 Hz), 7.62-7.59 (m, 2H), 7.40 (s, 1H), 7.35 (d, 2H, J=9.0 Hz), 7.25 (d, 2H, J=8.9 Hz), 7.05 (s, 1H).

LCMS Rₜ 11.72 min., purity 93%, m/z=350.2, no ionization.

### EXAMPLE 58

### Compound 227 3-bromo-N-(4-imidazol-1-yl-phenyl)-benzenesulfonamide

Synthesised ac cording to sulfonyl chloride coupling procedure 1 from the respective sulfonyl chloride and primary amine. LCMS Rₜ 11.20 min.; purity 95.0%; MS m/z 379.9 [M+H]⁺.

¹H NMR (300 MHz, CDCl₃) δ 8.02 (br s, 1H), 7.92 (dd, 1H, J=9.0 Hz), 7.72 (dt, 2H, J=2.5, 7.5 Hz), 7.46-7.36 (m, 4H), 7.22 (d, 2H, J=7.7 Hz), 7.09 (s, 1H).

### EXAMPLE 59

### Compound 241 N-[4-(dimethylamino)phenyl]-3-(2H-tetrazol-5-yl)benzenesulfonamide

To a solution of 3-cyano-N-[4-(dimethylamino)phenyl]benzenesulfonamide (500 mg, 1.66 mmol) in DMF (2.5 ml) was added sodium azide (119 mg, 1.82 mmol) and NH₄Cl (9 mg, 0.166 mmol). The mixture was heated at 125° C. for 18 hours, cooled and concentrated in vacuo. The residue was dissolved in H₂O (100 ml), filtered, extracted with AcOEt (3*100 ml), pH adjusted to 7 and the compound salted out of the aqueous layer. The light brown solid was dried in vacuo, 25 mg dissolved in methanol (0.5 ml) and purified by reverse phase preparative tlc plate (MeOH: H₂O 1:1) to provide (compound 241) (7 mg) as a light beige solid.

¹H NMR (300 MHz, d₃ MeOD) δ8.42 (s, 1H), 8.20-8.17 (m, 1H), 7.60-7.47 (m, 2H), 6.88 (d, 2H, J=9.02 Hz), 6.58 (d, 2H, J=8.97 Hz), 2.81 (s, 6H).

LCMS Rₜ 7.76 min.; purity 95%; MS m/z 345.3 [M+H]⁺.

### EXAMPLE 60

### Compound 242 2,4-dichloro-N-(1,2-dimethyl-1H-indol-5-yl)-N-methyl-benzenesulfonamide

Compound 161 was methylated according to methylation procedure 3 and purified by flash chromatography. Yield: 42%

¹H NMR (300 MHz; CDCl₃) δ 7.69 (d, 1H, J=8.5 Hz), 7.52 (m, 1H), 7.16 (dd, 1H, J=2.0 and 8.6 Hz), 7.12 (d, 1H, J=8.7 Hz), 6.93 (dd, 1H, J=2.0 and 8.7 Hz), 6.17 (m, 1H), 3.62 (s, 3H), 3.48 (s, 3H), 2.39 (s, 3H).

LCMS Rt 19.70 min.; purity 87.7%; no ionization

### EXAMPLE 61

### Compound 243 2,4-dichloro-N-methyl-N-(2-methyl-1H-indol-5-yl)-benzenesulfonamide

Compound 131 was methylated according to methylation procedure 3 and purified by flash chromatography. Yield: 29%

¹H NMR (360 MHz; CDCl₃) δ 7.90 (br, 1H), 7.70 (d, 1H, J=8.6 Hz), 7.53 (m, 1H), 7.19-7.14 (m, 2H), 6.89 (dd, 1H, J=2.0 and 8.6 Hz), 6.15 (m, 1H), 3.48 (s, 3H), 2.42 (s, 3H).

LCMS Rt 18.65 min.; purity 91.0%; no ionization

### EXAMPLE 62

### Compound 282 4′-fluoro-biphenyl-3-sulfonic acid (4-dimethylaminophenyl)-methylamide

Reaction was carried out according to procedure 1 for Suzuki coupling, with 2 equivalents of boronic acid. LCMS shows no remaining bromosulfonamide. Aqueous sodium hydrogen carbonate was used in place of water in the procedure above. Purification by prep HPLC.

¹H NMR (300 MHz, CDCl₃) δ 7.73 (d, 1H, J=7.3 Hz), 7.66-7.65 (m, 1H), 7.60-7.40 (m, 4H), 7.11 (t, 2H, J=8.7 Hz), 6.94 (d, 2H, J=9.1 Hz), 6.61 (d, 2H, J=9.0 Hz), 3.16 (s, 3H), 2.95 (s, 6H).

LCMS Rₜ 16.2 mins., purity=98%, m/z=385.2.

### EXAMPLE 63

### Compound 283 2-chloro-4-trifluoromethyl-N-[4-(2,6,6-trimethyl-4-oxo-4,5,-6,7-tetrahydro-indol-1-yl)-phenyl]-benzenesulfonamide

The reaction was carried out as described in procedure 2 for sulfonylation. No purification was necessary.

¹H NMR (300 MHz, CDCl₃) δ 8.24 (d, 1H, J=8.3 Hz), 7.81 (m, 1H), 7.71 (s, 1H), 7.64-7.68 (m, 1H), 7.29-7.26 (m, 2H), 7.11-7.08 (m, 2H), 6.33 (s, 1H), 2.33 (s, 2H), 2.27 (s, 2H), 1.93 (s, 3H), 1.02 (s, 6H).

LCMS Rₜ=14.1 min., purity=91%, m/z=511.3.

### EXAMPLE 64

### 1-methyl-6-aminoindole

6-Nitroindole was methylated as described in methylation procedure 1, reduced with hydrazine and Raney nickel as previously.

¹H NMR (300 MHz, CDCl₃) δ 7.42-7.39 (1H, m), 6.86 (1H, m), 6.61-6.56 (2H, m), 6.37 (1H, m), 3.68 (3H, s), 3.61 (2H, br).

### EXAMPLE 65

### Compound 284 4′-fluoro-biphenyl-3-sulfonic acid (1-methyl-1H-indol-6-yl)-amide

1-methyl-6-aminoindole was coupled using sulfonyl chloride coupling procedure 2a and reacted with 4-fluoroboronic acid as in Suzuki coupling procedure 1 and purified by flash chromatography. Yield 59%

¹H NMR (300 MHz; CD₃OD) δ 7.69-7.66 (m, 3H), 7.51-7.47 (m, 1H), 7.38-7.27 (m, 3H), 7.12-7.03 (m, 4H), 6.75-6.70 (m, 1H), 6.36-6.34 (m, 1H), 3.64 (s, 3H).

LCMS Rt 17.27 min.; purity 92.9%; MS m/z 380 [M]⁻

### EXAMPLE 66

### Compound 285 4′-fluoro-biphenyl-3-sulfonic acid (1-methyl-1H-indol-5-yl)-amide

5-Nitroindole was methylated as described in methylation procedure 1, reduced with hydrazine and Raney nickel as above, coupled using sulfonyl chloride coupling procedure 2a and reacted with 4-fluoroboronic acid as in Suzuki coupling procedure 1 and purified by flash chromatography. Yield 79%

¹H NMR (300 MHz; CD₃OD) δ 7.66-7.64 (m, 3H), 7.45 (d, 1H, J=8.0 Hz), 7.33-7.25 (m, 3H), 7.17 (d, 1H, J=8.7 Hz), 7.11-7.01 (m, 3H), 6.90-6.87 (m, 1H), 6.30 (m, 1H), 3.69 (s, 3H).

### EXAMPLE 67

### Compound 239 3-(5-acetylthien-2-yl)-N-[4-(dimethylamino)phenyl]benzenesulfonamide

To a solution of 3-bromo-N-[4-(dimethylamino)phenyl]benzenesulfonamide (100 mg, 0.28 mmol) in degassed DMF (10 ml) was added 5-acetyl-2-thienyl boronic acid (72 mg, 0.422 mmol), K₂CO₃ (117 mg, 0.845 mmol), palladium (II) acetate (7 mg, 0.028 mmol) and H₂O (57 μl, 3.19 mmol). The reaction was stirred at room temperature for 18 hours. The reaction was diluted with DCM (20 ml) and washed with saturated aqueous NH₄Cl (30 ml), H₂O (30 ml), brine (30 ml), dried (Na₂SO₄) and concentrated in vacuo. Half the residue was purified by cartridge column chromatography (AcOEt:cyclohexane 7:3) to provide (compound 239) (5 mg) as a green solid.

¹H NMR (300 MHz, CDCl₃) δ 7.89-7.87 (m, 1H), 7.76-7.73 (m, 1H), 7.66-7.61 (m, 2H), 7.45 (t, 1H, J=7.83 Hz), 7.26 (d, 1H, J=3.95 Hz), 6.91 (d, 2H, J=8.98 Hz), 6.67 (br s, 1H), 6.57 (d, 2H, J=8.88 Hz), 2.89 (s, 6H), 2.58 (s, 3H).

LCMS Rₜ 10.07 min.; purity 94%; MS m/z 401.2 [M+H]⁺.

### EXAMPLE 68

### Compound 277 5-chloro-thiophene-2-sulfonic acid [4-(4,6-dimethoxypyrimidin-2-yl)-phenyl]-amide

Synthesised according to sulfonyl chloride coupling procedure 1 (N.B. the reaction was carried out in the absence of tertiary amine) and purified by flash chromatography to provide compound 277 as an off-white solid.

¹H NMR (300 MHz, CDCl₃): δ 8.40 (br d, 2H, J=8.81 Hz), 7.32 (d, 1H, J=4.06 Hz), 7.22 (br d, 2H, J=8.81 Hz), 6.83 (d, 1H, J=4.05 Hz), 5.95 (s, 1H), 7.16 (br d, 2H, J=8.73 Hz), 4.02 (s, 6H).

LCMS Rₜ 15.69 min.; purity 97%; MS m/z 412 [M+H]⁺.

### EXAMPLE 69

### Compound 286 5-oxazol-5-yl-thiophene-2-sulfonic acid [4-(4,6-dimethoxypyrimidin-2-yl)-phenyl]-amide

Synthesised according to sulfonyl chloride coupling procedure 1 (N.B. the reaction was carried out in the absence of tertiary amine) and purified by flash chromatography to provide compound 286 as an off-white solid.

¹H NMR (300 MHz, CDCl₃): δ 8.42-8.37 (m, 2H), 7.32 (d, 1H, J=4.06 Hz), 8.27 (d, 1H, J=1.89 Hz), 7.50 (d, 1H, J=3.98 Hz), 7.34 (d, 1H, J=3.98 Hz), 7.28-7.21 (m, 2H), 6.47 (d, 1H, J=1.88 Hz), 5.94 (s, 1H), 4.01 (s, 6H).

LCMS Rₜ 14.86 min.; purity 96%; MS m/z 445 [M+H]⁺.

### EXAMPLE 70

### Compound 316 5-chloro-4-(4-fluoro-phenyl)-thiophene-2-sulfonic acid (4-dimethylamino-phenyl)-amide

To a degassed mixture of toluene (2 ml), ethanol (2 ml) and 2M aqueous Na₂CO₃ (2 ml) was added 4-bromo-5-chloro-N-[4-(dimethylamino)-phenyl]thiophene-2-sulfonamide (50 mg, 0.126 mmol), aryl boronic acid (0.139 mmol) and tetrakis(triphenylphosphine) palladium(0) (7.3 mg, 5 mol %). The mixture was heated at 90° C. for 18 hours. The reaction was cooled, filtered through celite and the celite cake washed with AcOEt (3*50 ml). The organic layer was dried (Na₂SO₄) and concentrated in vacuo. The residue was purified by prep HPLC to yield:

Suzuki procedure 5. Provided (compound 316) (8.98 mg) as a brown oil.

¹H NMR (300 MHz, CDCl₃) δ7.43-7.37 (m, 2H), 7.29 (s, 1H), 7.13-7.01 (m, 4H), 6.63 (d, 2H, J=8.51 Hz), 6.44 (br s, 1H), 2.94 (s, 6H).

LCMS Rₜ 16.36 min.; purity 96%; MS m/z 411.2 [M+H]⁺.

### EXAMPLE 71

### Compound 324 N-benzo[1,3]dioxol-5-yl-2,4-dichloro-N-methyl-benzenesulfonamide

Compound 157 was methylated according to methylation procedure 2 and purified by flash chromatography. Yield: 64%

¹H NMR (300 MHz; CDCl₃) δ 7.79 (d, 1H, J=8.6 Hz), 7.52 (d, 1H, J=2.0 Hz), 7.31-7.24 (m, 1H), 6.71-6.59 (m, 3H), 5.95 (s, 2H), 3.36 (s, 3H).

LCMS Rt 17.56 min.; purity 98.2%; no ionization.

1. A sulphonamide derivative of formula (I) or a physiologically acceptable salt thereof, 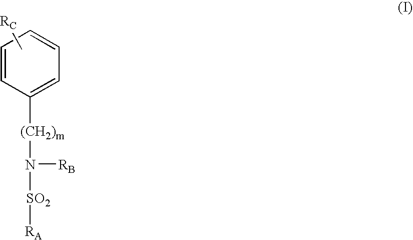
where
R_{C} is an optionally substituted 4-6-membered heterocyclic ring containing one or more N atoms, or
R_{C} forms together with the phenyl ring to which it is attached a benzodioxolyl group, or
R_{C} is —NR¹R², where
R¹ is hydrogen or alkyl,
R² is alkyl or an optionally substituted 4-6-membered heterocyclic ring containing one or more N atoms, or
R¹ and R² taken together with the nitrogen atom to which they are attached form a heterocyclic group, which may contain one or more additional heteroatoms selected from O and N and which may be substituted, or
R¹ and R² are absent and the nitrogen atom together with the adjacent carbon atom forms a heterocyclic ring, which may contain one or more additional heteroatoms selected from N, O and S and which may be substituted, provided that the nitrogen atom together with the benzene moiety does not form an isoquinoline or an indol-7-yl ring,
m is 0 or 1,
R_{A} is a group having the formula 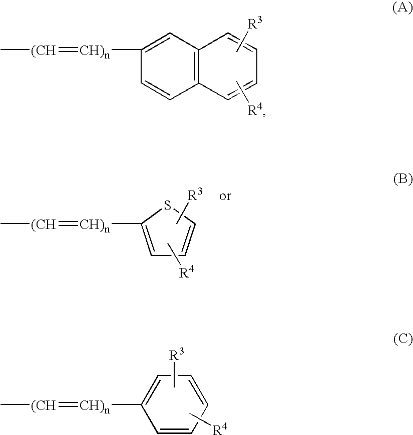
wherein
n is 0,
R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluorometoxy, an optionally substituted aryl or heterocyclic group, and
R_{B} is hydrogen or alkyl.

2. A derivative according to claim 1 where R¹ and R² represent methyl, R³ is 2-chloro and R⁴ is 4-chloro.

3. A derivative according to claim 1 where R¹ is hydrogen, R² is 4,6-dimethylpyrimidin-2-yl, R³ is chloro and R⁴ is chloro.

4. A derivative according to claim 1 where R¹ and R² represent methyl, R³ is hydrogen and R⁴ is 3,4-dimethoxyphenyl.

5. A derivative according to claim 1 where R¹ and R² represent methyl, R³ is hydrogen and R⁴ is 4-fluorophenyl.

6. A derivative according to claim 1 where R¹ and R² represent methyl, R³ is hydrogen and R⁴ is bromo.

7. A derivative according to claim 1, which is 4′-fluoro-biphenyl-3-sulfonic acid benzo[1,3]dioxol-5-ylamide.

8. A derivative according to claim 1, which is 4′-fluoro-biphenyl-3-sulfonic acid (2-methyl-benzooxazol-6-yl)-amide.

9. A derivative according to claim 1, which is 2,4-dichloro-N-(1,2-dimethyl-1H-indol-5-yl)-N-methyl-benzenesulfonamide.

10. A derivative according to claim 1, which is 4′-fluoro-biphenyl-3-sulfonic acid (4-dimethylaminophenyl)-methyl-amide.

11. A derivative according to claim 1, which is N-[4-(dimethylamino)phenyl]-4′-fluoro-2′-methyl-1,1′-biphenyl-3-sulfonamide.

12. A derivative according to any of claims 1 to 11 for use as an inhibitor for collagen receptor integrins.

13. A derivative according to any of the claims 1 to 11 for use as an inhibitor for α2β1 integrin.

14. A derivative according to any of claims 1 to 11 for use as an α2β1 integrin I domain inhibitor.

15. A derivative according to any of claims 1 to 11 or a physiologically acceptable salt thereof for use as a medicament.

16. A derivative according to claim 15 for use as a medicament for treating thrombosis and cancer spread.

17. The use of a derivative according to any of claims 1 to 11 or a physiologically acceptable salt thereof for preparing a pharmaceutical composition for treating disorders relating to thrombosis and cancer spread.

18. A pharmaceutical composition comprising an effective amount of a derivative according to any of claims 1 to 11 or a physiologically acceptable salt thereof in admixture with a pharmaceutically acceptable carrier.

19. A process for preparing a benzene sulphonamide according to claim 1, comprising reacting a compound of formula (II) 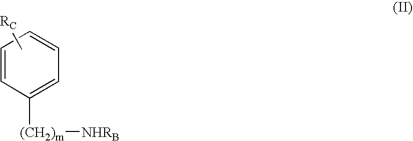
where R_{B}, R_{C} and m are as defined above, with a compound of formula (III)
R_{A}—SO₂hal (III)
where R_{A} is as defined above and hal is halogen.
